(19) United States
(12) Patent Application Publication
(10) Pub. No.: US 2023/0381234 A1
(43) Pub. Date: Nov. 30, 2023

(54) COMBINATION OF ADOPTIVE CELL THERAPY AND CHEMOTHERAPY FOR ACUTE MYELOID LEUKEMIA

(71) Applicant: LOMA LINDA FACULTY MEDICAL GROUP, Loma Linda, CA (US)

(72) Inventors: Yi (David) Xu, Loma Linda, CA (US); Huynh Cao, Loma Linda, CA (US)

(73) Assignee: LOMA LINDA FACULTY MEDICAL GROUP, Loma Linda, CA (US)

(21) Appl. No.: 18/249,154

(22) PCT Filed: Oct. 14, 2021
(86) PCT No.: PCT/US21/71892

§ 371 Date: Apr. 14, 2023

Related U.S. Application Data
(60) Provisional application No. 63/198,386, filed on Oct. 14, 2020.

(51) Int. Cl. A61K 35/17; A61K 31/706; C07K 16/28; A61P 35/02

(52) U.S. Cl. CPC A61K 35/17; A61K 31/706; C07K 16/2818; A61P 35/02

(57) ABSTRACT

Disclosed here are methods of treatment of cancers by administering autologous tumor infiltrating lymphocytes optionally in combination with chemotherapeutic and immunotherapeutic agents. Methods include treating acute myeloid leukemia with a combination of autologous TILs-based adoptive cell therapy and agents, such as a PD-1 inhibitor and a hypomethylating agent. The TILs can be bioengineered to modify the expression of function of a gene or a molecule of interest.

### CROSS-REFERENCE TO RELATED APPLICATIONS

This application claims the benefit of priority to U.S. Provisional Application No. 63/198,386, filed on Oct. 14, 2020, which is incorporated herein by reference in its entirety.

### TECHNICAL FIELD

The disclosure relates to compositions and methods of treatment of cancers utilizing an autologous TIL-based adoptive cell therapy. This disclosure also related to compositions and methods of treatment of cancers utilizing a combination of bioengineered tumor infiltrating lymphocytes (TILs) and chemotherapeutic agents.

### BACKGROUND

Acute myeloid leukemia (AML) is a hematopoietic cancer that involves a complex interplay between different cells of the bone marrow, cytokines, growth factors, and neural modulation. AML most commonly occurs in older adults. With multiple subtypes and highly heterogeneous genetic abnormalities, AML is extremely difficult to treat with the current chemotherapeutic regimens. AML relapse caused by chemotherapy-resistant leukemia stem cells (LSCs) is common, and remains one of the most important causes of treatment failure. There is a need for less toxic and more effective treatment regimens for AML. Cancer immunotherapy utilizes components of the immune system to eliminate cancer cells while sparing healthy cells. Bioengineering T cells including generating Chimeric Antigen Receptor T cells (CAR-T) is a recent approach to provide the precise immunotherapy to each cancer patient. Recently, tumor infiltrating lymphocytes (TILs) or marrow-infiltrating lymphocytes (MILs) have been used to treat solid tumors and multiple myeloma. In contrast to the difficulties of identifying LSCs-specific antigens (Ag) in CAR-T therapies, TILs have the advantage of being capable of sensing various signals from tumor microenvironment and being Ag-specific by directly targeting mutated peptides of cancer stem cells. However, complications including the deficiency, dysfunction and exhaustion of TILs in the cancer microenvironment arise.

### SUMMARY

Disclosed herein are compositions and methods addressing the shortcomings of the art, and may provide any number of additional or alternative advantages. Disclosed here are methods of treatment of cancers by administering an autologous TIL-based adoptive cell therapy. In certain embodiments, the methods of treatment of cancers include administration of one or more of TILs and chemotherapeutic agents. Embodiments of the methods include treating AML with a combination of TIL-based cell therapy and chemotherapeutic agents. The chemotherapeutic agents include one or more of a hypomethylating agent and inhibitor of programmed cell death protein 1 (PD-1). Embodiments of the methods include treating AML with a combination of TIL-based cell therapy, a chemotherapeutic agent, and an immunotherapeutic agent. In an embodiment, the treatment regimen for an AML patient includes a TILs-based adoptive cell therapy along with administration of one or more inhibitors of PD-1 and AZA. The PD-1 inhibitor can be a chemical compound or an immunotherapeutic agent, e.g., an anti-PD-1 antibody, such as nivolumab, dostarlimab, pembrolizumab, and cemiplimab. In an embodiment, a relapse of AML is managed or prevented by administering a TILs-based adoptive cell therapy along with administration of one or more PD-1 inhibitors and AZA. In an embodiment, the method of treatment of AML includes a chemo-immunotherapy regimen of ex vivo expanded autologous PD-1-inhibited-TILs, AZA, and one or more PD-1 inhibitors.

In some aspects, the methods of treating AML in a subject include the steps of isolating tumor infiltrating lymphocytes (TILs) from the subject, culturing the isolated TILs in a culture system to produce cultured TILs, and administering the cultured TILs to the subject. Accordingly, the present disclosure provides an autologous TIL-based adoptive cell therapy to treat AML. The TILs can be isolated from bone marrow samples of the subject, peripheral blood samples of the subject, or the combination thereof. In some embodiments, the isolated and/or cultured TILs include naïve T cells (e.g., CD3⁺ T cells). In some embodiments, the isolated and/or cultured TILs include CCR7⁺CD95⁻ and/or CD62L⁺CD45RA⁺ T cells. In some embodiments, TILs are expanded ex vivo, i.e., the number of TILs is increased ex vivo. In some embodiments, CD3⁺ TILs are expanded ex vivo by about a 1000-fold or by about three log-fold from a sample from the subject. In some embodiments, TILs are cultured ex vivo in the presence of a PD-1 inhibitor, e.g., an anti-PD-1 antibody, such as nivolumab, dostarlimab, pembrolizumab, and cemiplimab. In some embodiments, the number of CD8⁺ TILs are increased in the step of culturing the TILs. In some embodiments, the methods of the present disclosure further include bioengineering TILs or T cells isolated from the subject. In some embodiments, the TILs are bioengineered to increase expression or function of a 25-hydroxyvitamin D-1 alpha hydroxylase (CYP27B1) gene. In some embodiments, the TILs are bioengineered to suppress expression or function of a PD-1 gene. In some embodiments, the TILs are bioengineered to improve antigen-specificity. In some embodiments, the TILs are engineered to increase expression or function of one or more of CD3, CCR7, CD62L, CD45RA, CD95, CD127, CD27, and CD28. In some embodiments, the methods of the present disclosure further include administering a therapeutically effective amount of a PD-1 inhibitor, e.g., nivolumab, dostarlimab, pembrolizumab, or cemiplimab, and a hypomethylating agent, e.g., 5-azacytidine (AZA). The autologous TIL-based therapy has several advantages, including the use of autologous transplantation avoids graft-versus-host disease, addresses the issue of antigen-specificity, allows reprogramming of the TILs in vitro, and enhances immediate T cell immunity in vivo.

Numerous other aspects, features and benefits of the present disclosure may be made apparent from the following detailed description taken together with the figures. The compositions can include the cell-based compositions described herein along with one or more chemotherapeutic and immunotherapeutic components, or ingredients depending on desired prevention and treatment goals. It should be further understood that both the foregoing general description and the following detailed description are exemplary and explanatory and are intended to provide further explanation of the various inventions as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the figures of the accompanying drawings.

FIG. 1 is an illustration of the mechanisms for treatment for AML patients, according to embodiments of this disclosure.

FIGS. 2A-2C are density plots from FACS analysis for the expressions of PD-1 and cluster of differentiation protein 3 (CD3).

FIGS. 3A-3B are density plots from FACS analysis for the expressions of CD8 and CD3 in TILs in culture. FIG. 3C is a graphical representation of the percentage of cells that express CD8 and CD3 at Day 0 and Day 5. FIG. 3D is a graphical representation of the number of CD3⁺ cells at Day 1 and Day 21. FIG. 3E presents representative images of 2 AML patients' TIL culture; *P<0.05.

FIGS. 4A-4F provide characterization of the bioengineer-expanded TILs lentivirally and pharmaceutically ex vivo. FIG. 4A is a FACS plot of GFP expression after lentiviral transduction into TILs. FIGS. 4B and 4C are FACS plots of PD-1 expression in CD3⁺TILs with or without treatment of an inhibitor of PD-1 expression, Nivolumab. FIG. 4D is a graphical representation of the percentage of CD3⁺TILs that express PD-1 with or without treatment using Nivolumab. FIG. 4E is a FACS plot to measure cytotoxicity following no treatment, treatment with CD3⁺TILs with or without exposure to Nivolumab. FIG. 4F is a graphical representation of the percentage of viable blast count in the cytotoxic tests. N=3.

FIGS. 5A-5E are directed to the generation of femoral patient-derived xenograft (PDX) AML mouse model. FIG. 5A is an illustration of the femoral PDX AML mouse model. FIG. 5B is an X-ray image of injection. FIGS. 5C, 5D, and 5E are bioluminescence images of Luciferase-GFP⁺ AML blasts in vitro and in vivo inside the bone marrow of AML mice, respectively. FIG. 5F is a schematic illustration depicts protocols for ex vivo expansion of PD-1-inhibited TILs and in vivo evaluation of immunotherapy in PDX AML mouse models.

FIGS. 6A1-6A4 and FIGS. 6B1-6B2 are photographic images of femoral bone marrow in naïve environment and when transplanted PD-1-inhibited-TILs, respectively.

FIGS. 7A-7B are graphical representations of RNA-seq analysis of AML cells treated with single agents-1.25(OH)2D3 and AZA and their combination. FIG. 7A is a graphical representation of expression of human VDR and FIG. 7B is a graphical representation of VDR expression in cells treated with single agents-1.25(OH)2D3 and AZA and their combination as confirmed by quantitative PCR (qPCR) experiments.

FIG. 8 is an illustration of bioengineered-TILs based adoptive cell therapy for AML, according to an embodiment.

FIGS. 9A-9C depict immunophenotyping of TILs from AML patients' bone marrow (BM) samples. FIG. 9A shows density plots from FACS analysis for expression of PD-1 and CD3. FIGS. 9B and 9C are graphical representations of the percentages of CD3⁺ T cells and PD-1⁺ CD3⁺ T cells in two groups of patients with low and high numbers of CD3⁺ T cells or PD-1⁺ CD3⁺ T cells, respectively.

FIGS. 10A-10F depict expansion of high number CD3⁺TILs ex vivo. FIG. 10A is an illustration of the experimental procedures of ex vivo culture of isolated CD3⁺ TILs. FIGS. 10B and 10C are representative FACS plots showing the percentage data of CD3⁺ or CD8⁺ cells on day 0 and day 5. FIG. 10D is a graphical representation of the cumulative FACS percentage data of CD8⁺CD3⁺ T cells on day 0 and day 5. FIGS. 10E and 10F are representative FACS plots showing the PD CD8⁺ cells on day 0 and day 5. Where applicable, data are means±SEM and were analyzed by Student t-test. ** P<0.01.

FIGS. 11A-11D depict expansion of low number CD3⁺ TILs ex vivo. FIG. 11A is an illustration of the experimental procedures of ex vivo culture of isolated CD3⁺ TILs. FIG. 11B is a representative FACS plots showing the CD4⁺ CD3⁺ or CD8⁺ CD3⁺ T cell subsets and their PD-1 expression on day 7. FIG. 11C is a graphical representation of the cumulative FACS percentage data of CD3⁺ T cells on day 21. FIG. 11D are representative FACS plots showing the CD4⁺ CD3⁺ or CD8⁺ CD3⁺ T cell subsets and their PD-1 expression on day 21. Where applicable, data are means±SEM and were analyzed by Student t-test. **P<0.01

FIG. 12A-12E depict bioengineering of primary AML TILs ex vivo. FIGS. 12A and 12B are representative FACS plots showing the percentage data of PD-1 expression in CD3⁺ TILs with or without treatment of Nivolumab. FIG. 12C is a graphical representation of the cumulative FACS percentage data of PD-1 expression in CD3⁺ TILs with or without treatment of Nivolumab; N=3. FIG. 12D is a representative FACS plot of PD-L1 expression in CD117⁺ AML blasts. FIG. 12E is a graphical representation of the FACS analyses of GFP expression in PD-1Negative-CYP27B1⁺TILs after lentiviral transduction. Where applicable, data are means±SEM and were analyzed by Student t-test. * P<0.05.

FIGS. 13A-13J depict correlation of the existence of naïve TILs in AML patients' BM samples and their proliferation capabilities ex vivo. FIGS. 13A, 13E, and 13F represent “no growth” TILs (from patients #11, 12, 19). FIGS. 13B and 13I represent “slow growth” TILs (from patient #13). FIGS. 13C, 13G, 13H, and 13J represent “quick growth” TILs (from patients #14-18, 20). FIGS. 13A-13C are representative phase-bright images showing different growth of CD3⁺ TILs; Scale bar: 100 μm. FIG. 13D is a graphical representation of the cumulative FACS percentage data of CCR7⁺ CD95-TILs between No growth and Quick growth. FIG. 13E shows a representative FACS plots of TILs with no growth from Patient #12. Red arrow indicates CCR7⁺ CD95− cells. FIG. 13F shows a representative FACS plots of TILs with no growth from Patient #11. Short red arrow indicates CCR7⁺ CD95⁻ cells; Long red arrow indicates CCR7⁺ CD95⁻ CD62L⁺CD45RA⁺ cells. FIG. 13G shows representative FACS plots of TILs with quick growth from Patient #14; short green arrow indicates CCR7⁺ CD95− cells. FIG. 13H shows representative FACS plots of TILs with quick growth from Patient #15. Short green arrow indicates CCR7⁺CD95⁻ cells. Long green arrow indicates CCR7⁺CD95⁻ CD62L⁺CD45RA⁺ cells. FIG. 13I shows representative FACS plot of TILs with slow growth from Patient #13; Brown arrow indicates CCR7⁺CD95⁻ cells. FIG. 13J shows representative FACS plot of TILs with quick growth from Patient #16; Green arrow indicates CCR7⁺CD95⁻ cells. In each of the foregoing figures, # indicates Patient No; Red X mark indicates no growth; Brown Star mark indicates slow growth; Green Star Mark indicates quick growth.

FIGS. 14A-14B depict comparison of CD62L⁺CD95+ TILs in the AML Patients' bone marrow mononuclear cells (BM-MNC). FIG. 14A shows representative FACS plots showing the percentage data of CD62L⁺CD45RA⁺ TILs from different AML patient BMMNC; Red patient numbers: no growth; Brown patient numbers: slow growth; Green patient numbers: quick growth. FIG. 14B is a graphical representation of the cumulative FACS percentage data of CD62L⁺CD45RA⁺TILs between No/Slow growth and Quick growth. Where applicable, data are means±SEM and were analyzed by Student t-test.

FIGS. 15A-15B depict comparison of naive T cells and differentiated T cells in bone marrow and peripheral blood of same patients (#11-#16). FIG. 15A shows representative FACS plots of naïve T cells for CCR7 and CD95 expression. FIG. 15B shows representative FACS plots of differentiated T cells for CD4 and CD8 expression.

FIGS. 16A-16C depict ex vivo cytotoxic tests of peripheral blood T cells (PB-T cells) and TILs from bone marrow (BM-TILs) from a patient having “quick growth” TILs (#14). FIG. 16A shows representative FACS plots showing the percentage of viable AML blasts in the cytotoxic test; The T cells versus blasts ratio equals 10:1. FIG. 16B is a graphical representation of the cumulative FACS percentage data of viable AML blasts. FIG. 16C shows representative FACS plots showing the percentage of viable CD8 percentage. Where applicable, data are means±SEM and were analyzed by Student t-test. **P<0.01, N=3.

FIGS. 17A-17C depict Transplantation of AML TILs in vivo. FIG. 17A is an illustration of the experimental procedures of transplanting TILs in vivo. FIG. 17B shows representative immunohistochemical images showing the colocalization of Qtracker 655⁺ TILs (red) with CD3 expression (green) in the bone marrow of naïve mice. DAPI: blue nuclei. FIG. 17C shows representative Immunohistochemical images showing Qtracker 655⁺ TILs (red) located close to AML blasts (GFP-labeled, green) in the bone marrow of AML mice on day 10.

### DETAILED DESCRIPTION

AML is a hematopoietic cancer that has a heterogeneous cell population and is an aggressive malignancy with poor prognosis. AML most commonly occurs in older adults. With multiple subtypes and heterogeneous genetic abnormalities, AML is extremely difficult to treat with the current chemotherapeutic regimens, and has the lowest survival rate amongst all types of leukemia. Disease relapse is the most important cause of treatment failure. The standard therapy for AML involves intensive chemotherapy induction with combination of daunorubicin or idarubicin and cytarabine. However, many elderly patients cannot tolerate such regimens. The mortality of elderly AML (at age or older), following relapse, remains at 95% within 5 years, and this has not changed much over the last 30 years. Therefore, there is a need for novel targeted therapies that are more active and less toxic for this patient population. Single agent 5-Azacytidine (AZA) is not effective in inducing remission, possibly due to the development of increased expression of PD-1/PD-L1. Leukemia stem cells (LSCs) are a key factor in the problem of relapse. These LSCs are characterized by self-renewal, cell-cycle quiescent and chemotherapy-resistance. Unfortunately, AZA-based cytotoxic therapy fails to completely eliminate the LSCs.

The programmed cell death 1 (PD-1) receptor is expressed on activated T cells, B cells, macrophages, regulatory T cells (Tregs), and natural killer (NK) cells. PD-1 inhibitors and PD-L1 inhibitors are checkpoint inhibitors that interfere with the interaction between programmed cell death protein 1 (PD-1) and its B7 family of ligands, programmed death-ligand 1 (PD-L1 or B7-H1) or PD-L2 (B7-DC). Activation of PD-1/PD-L1 signaling serves as a principal mechanism by which tumors evade antigen-specific T-cell immunologic responses. Blocking either PD-1 or PD-L1 enhances T-cell-mediated anti-tumor activity. Cancer immunotherapy utilizes components of the immune system to eliminate cancer cells while sparing healthy cells. Among immunotherapies, tumor infiltrating lymphocyte (TIL) adoptive cell therapy is a therapy in which TILs, which are primarily T-cells, are isolated from patients' surgically removed tumors, primed ex vivo, and reintroduced into patients to eliminate tumor cells. As used herein, “tumor infiltrating lymphoycytes” (TILs) are anti-tumor lymphocytes, i.e., lymphocytic cell populations that invade a tumor tissue and have the ability to recognize and kill tumor cells. In some aspects, TILs include characteristics of naïve T cells. In some embodiments, TILs include CD3⁺ T cells. In some embodiments, TILs include CCR7⁺CD95⁻ and/or CD62L⁺CD45RA⁺ T cells.

Addition of chemotherapeutic agents along with PD-1/PD-L1 inhibitors sensitizes tumors to respond to immunotherapies, and increases CD8⁺ T cell infiltration and cytotoxicity to tumors. PD-1 inhibitors and PD-L1 inhibitors activate immune responses from TILs, resulting in a reduction of tumor metastasis and growth.

The present disclosure describes various embodiments related to compositions and methods for management or treatment of cancers, such as AML, gastric cancer, or breast cancer. In the following description, numerous details are set forth in order to provide a thorough understanding of the various embodiments. Before the present methods and compositions are described, it is to be understood that these embodiments are not limited to particular methods or compositions described, as such may, of course, vary. It is also to be understood that the terminology used herein is for the purpose of describing particular embodiments only, and is not intended to be limiting, as the scope of the present embodiments will be limited only by the appended claims. The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

A “subject” refers an animal, such as a mammal, including a primate (such as a human, a non-human primate, e.g., a monkey) and a non-primate (such as a mouse). In some aspects of the invention, the subject is a human. In some aspects, the subject is a pediatric subject, such as a neonate, an infant, or a child. In other aspects, the subject is an adult subject.

A “patient” refers to a subject who shows symptoms and/or signs of a disease, is under treatment for disease, has been diagnosed with a disease, and/or is at risk of developing a disease. A “patient” can be human and veterinary subjects. Any reference to subjects in the present disclosure, should be understood to include the possibility that the subject is a “patient” unless clearly dictated otherwise by context. More specifically, the subject in certain aspects is a patient who has a liquid cancer, such as a leukemia.

As used herein, the terms “treating”, “treatment” and the like, shall include the management and care of a subject or patient for the purpose of combating a disease, condition, or disorder and includes the administration of a composition to prevent the onset of the symptoms or complications, alleviate the symptoms or complications, reduce at least one associated sign, symptom, or condition, or eliminate the disease, condition, or disorder. Treatment also refers to a prophylactic treatment, such as prevention of a disease (e.g., AML) or prevention of at least one sign, symptom, or condition associated with the disease (e.g., relapse of AML). Treatment can also mean prolonging survival as compared to expected survival in the absence of treatment.

Administration of TIL Adoptive Cell Therapy with a PD-1 Inhibitor and Hypomethylating Agent

Most AML patients experience relapse 2-3 years after complete remission. Besides the report of dysfunctional immunity in AML patients, the existence of leukemia stem cells (LSCs) is also a key factor in the problem of relapse. These LSCs are quiescent, chemo-resistant, and they are not wiped out easily. In an embodiment, autologous transplantation of sufficient amount of ex vivo expanded PD-1-inhibited-TILs are administered to replenish impaired immune system in AML patients. The genetically manipulated TILs can specifically target LSCs. The combination of TIL adoptive cell therapy with a PD-1 inhibitor and AZA can reduce the likelihood of relapse of AML at the early stage. AML blasts express PD-L1 and are susceptible to PD-1 inhibitor treatment. In an embodiment, a combination immunotherapy of PD-1-inhibited-TILs, AZA and a PD-1 inhibitor results in potent anti-leukemic effects, and overcomes the immune-escape of LSCs and AML blasts ex vivo and in vivo.

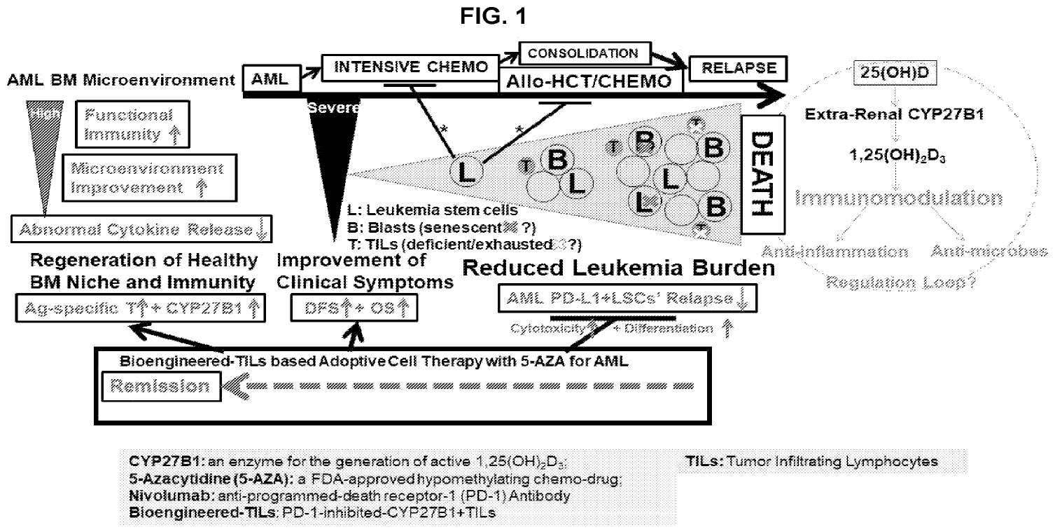

Methods disclosed herein include administration of TIL adoptive cell therapy with a PD-1 inhibitor and AZA to the AML BM microenvironment which can then improve survival and reduce the leukemia burden endured by patients. In an embodiment, a combination of TILs, PD-1 inhibitors, and AZA are administered to treat AML and prevent or mitigate a relapse. CD3⁺ T-cells are isolated from AML patients' bone marrow (BM) samples. In some embodiments, these cells express PD-1. These primary CD3⁺ T-cells could be isolated and expanded ex vivo. In an embodiment, the PD-1 expression is inhibited in TILs to restrict the inhibitory signal of blast cells on the CD3⁺ T-cell population and lead to more effective killing of LSCs and AML blasts. In an embodiment, the ex vivo expanded autologous TILs in combination with anti-PD-1 antibodies and AZA is administered to produce a superior anti-leukemic therapeutic effect against LSCs and AML blasts and enhance the disease-free and overall survival. In an embodiment, the ex vivo expanded autologous TILs are first exposed to an inhibitor of PD-1 to generate PD-1-inhibited-TILs. These PD-1-inhibited-TILs are administered in combination with AZA to generate a cytotoxic effect and treat cancer. In an embodiment, autologous PD-1-inhibited-TILs are administered in combination with AZA and a PD-1 inhibitor to reduce the leukemic burden and increase survival of an AML patient. These combinations of TILs with chemotherapeutic agents prevent relapse and improve AML patient survival outcomes. FIG. 1 is an illustration of the mechanisms for treatment for AML patients, according to embodiments of this disclosure.

A PD-1 inhibitor can be a chemical agent or a biological agent. A PD-1 inhibitor will inhibit the expression or function of PD-1 by TILs by at least 10% (e.g., at least 20%, at least 30%, at least 40%, at least 50%, at least 60%, at least 70%, at least 80%, at least 90%, 100%) as compared to expression or function of PD-1 in TILs that have not been treated by the PD-1 inhibitor. Examples of immunotherapeutic PD-1 inhibitors include one or more of atezolizumab, avelumab, cemiplimab, durvalumab, nivolumab, and pembrolizumab.

A “therapeutically effective amount” is an amount sufficient to effect desired clinical results (i.e., achieve therapeutic efficacy). A therapeutically effective dose can be administered in one or more administrations. “Administering” refers to the physical introduction of a therapeutic agent to a subject in need thereof. Exemplary routes of administration for agents to inhibit proliferation of mesenchymal cancer stem cells. include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route, or orally. Other non-parenteral routes include a topical, epidermal or mucosal route of administration, for example, intranasally, vaginally, rectally, sublingually or topically. Administering can also be performed, for example, once, a plurality of times, and/or over one or more extended periods. Therapeutic agents can be constituted in a composition, e.g., a pharmaceutical composition containing an antibody and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

In an embodiment, in addition to the treatment of the TILs with a PD-1 inhibitor ex vivo, to ensure the inhibition of PD-1 expression in TILs during their expansion, a PD-1 inhibitor is administered to the patient preceding or along with administration of transplantation of the TILs. In an embodiment, administration of AZA can start ahead of the TIL transplantation. In an embodiment, combination treatment with autologous PD-1-inhibited-TILs and AZA shows superior therapeutic efficacy and prolonged survival as compared to the single treatment with PD-1-inhibited-TILs or AZA alone. In an embodiment, AZA is administered preceding the administration of PD-1-inhibited-TILs to condition the BM in preparation for the transplantation. Thus, AZA will simultaneously serve as a treatment and an agent to prime the BM. In an embodiment, appropriate dose of IL2 is administered to help T cell proliferation in vivo.

In addition, cell vehicles for TILs adoptive cell transfer could be further manipulated to create antigen specific to make chimeric antigen receptor (CAR)-TILs as the precision medicine for all cancer patients. Embodiments include methods for ex vivo expansion of tumor-infiltrating lymphocytes for use in adoptive cell therapy. In one such method, the steps include: (a) obtaining autologous tumor-infiltrating lymphocytes from the subject, (b) culturing the lymphocytes in a culture medium containing several interleukins to produce expanded lymphocytes, and (c) administering the expanded lymphocytes, a PD-1 inhibitor, and AZA to the patient.

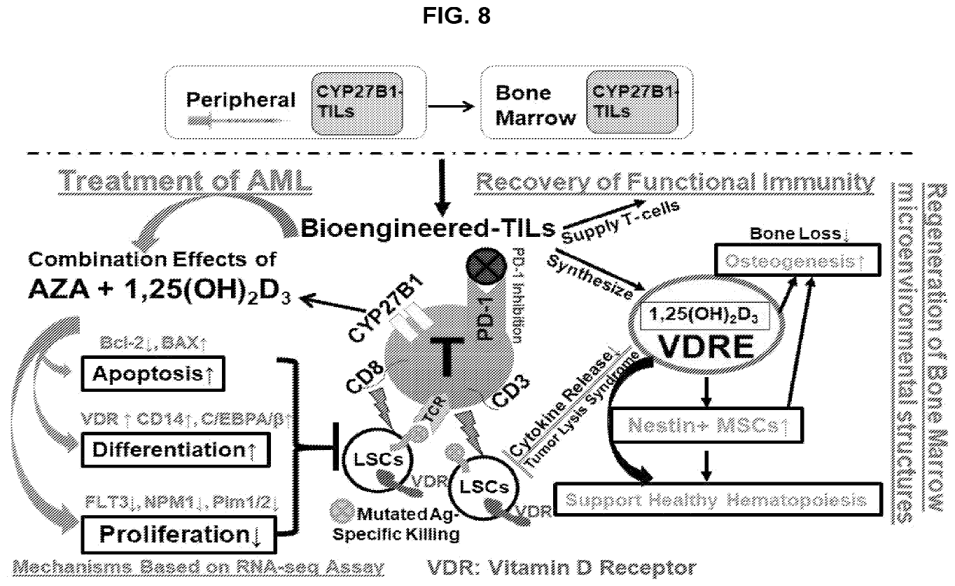

In an embodiment, the combination of autologous CYP27B1-expressing TILs and PD-1 inhibitors are administered to treat AML by elongating the overall survival without significant side effects. TILs are an alternative cell vehicle to deliver transgenes or peptides to AML BM. CYP27B1, also known as 25-Hydroxyvitamin Dl-alpha-hydroxylase, VD 1A hydroxylase, cytochrome p450 27B1, 1-alpha-hydroxylase, is a cytochrome P450 enzyme encoded by the CYP27B1 gene. CYP27B1 catalyzes calcifediol to calcitriol, i.e., the bioactive form of Vitamin D. Local newly synthesized Vitamin D by ectopic CYP27B1 will not only contribute to BM regeneration but also suppress tumor lysis syndrome. Because TILs have been sensitized by surrounding AML antigens before isolation, the antigen (Ag)-specific property will direct TILs to locate AML cells in the BM or the peripheral tissues and kill them in situ. In addition, treatment of PD-1 inhibitors will prevent negative signaling from AML cells exhausting TILs in vivo. Therapeutic roles of TILs in vivo and how Vitamin D regenerates the healthy microenvironment for remission are illustrated in FIG. 8. FIG. 8 is an illustration of bioengineered-TILs based adoptive cell therapy for AML, according to an embodiment.

CYP27B1-based vitamin D gene therapy provides several advantages. It enhances the differentiation of leukemic cancer cells because of vitamin D as a strong differentiator. It generates synergistic effect with 5-AZA, an FDA-approved epigenetic modulator to eliminate majorities of viable blasts. It serves as an immune-modulator to suppress Cytokine Release Syndrome, a major side effect of adoptive T cell therapies including CAR-T; and improves the regeneration of bone marrow microenvironment and healthy hematopoiesis through positively regulating stem cell properties of mesenchymal stem cells and hematopoietic stem cells. Further embodiments of TIL adoptive cell therapy are provided below.

Ex Vivo Isolation, Expansion, and Bioengineering of TILs from Bone Marrow or Peripheral Blood Samples of AML Subjects

In some aspects, methods of treating AML in a subject include the administration of an autologous TIL-based adoptive cell therapy. One such method includes the steps of isolating tumor infiltrating lymphocytes (TILs) from the subject, culturing the isolated TILs in a culture system to produce cultured TILs, and administering the cultured TILs to the subject. In some embodiments, TILs are CD3⁺ T cells that are isolated from bone marrow samples of the subject, peripheral blood samples of the subject, or both the bone marrow and peripheral blood samples of the subject. In some embodiments, CD3⁺ TILs are isolated, by e.g., using CD3 microbeads and a separator, e.g., MiniMACS™ Separator, with an MS Column. In some embodiments, selected CD3⁺ T cells comprise naïve T cells and are considered AML TILs.

In some embodiments, TILs, e.g., CD3⁺ T cells, isolated from peripheral blood samples and TILs, e.g., CD3⁺ T cells, isolated from bone marrow have similar characteristics with regards to cell surface molecule expression and/or cytotoxic function. In some embodiments, the use of peripheral blood samples as source of TILs provides several advantages. The peripheral blood-derived TILs can be acquired in large amount by leukapheresis and can be bioengineered (e.g., genetically engineered) to improve antigen-specificity.

In some embodiments, isolated TILs are cultured and expanded efficiently in the ex vivo culture system. In some embodiments, these isolated TILs are CD3⁺ T cells. The TILs are isolated and cultured at 37° C. and 5% CO₂ in a RPMI 1640 culture medium containing 10% fetal bovine serum, antibiotics, IL2, and Dynabeads® Human T-Activator CD3/CD28 without feeder cells. In some embodiments, the TILs are expanded following the stages set forth below:

Stage 1 (Naïve TILs): The cell density of CD3⁺ TILs can be started around 300 μl of 20,000 cells/ml in appropriate wells of 48-well-plates, according to the cell count after pulling down CD3⁺ TILs. Fresh media is added at 1:1 ratio to each well every 2 days, and the cells/media are mixed. Based primarily on the growth of the TILs, medium change is performed every 5-7 days, and split cells at the ratio 1:3.

Stage 2 (Ready to grow): After 7 days, IL7 (25 ng/ml) and IL15 (25 ng/ml) are added to the media with IL2 (1000 U/ml). Although every patient's bone marrow mononuclear cells (BM-MNC) sample are different, TILs are raised in 48-well-plates for expansion to sufficient amount during the beginning 10-14 days, instead of large wells and flasks.

Stage 3 (Quickly expand and differentiate into T effectors): After 10-14 days, TILs can grow very fast. Medium change is performed every 2 days, and the cells were split quickly to expanded TILs at the ratio 1:4 to 1:8. TILs can be expanded in 24 or 12-well-plates. Dynabeads® Human T-Activator CD3/CD28 can be used once every 2 weeks for re-stimulation of TILs.

In some embodiments, TILs are expanded ex vivo, with the number of the TILs increased to about 1.1-10-fold, about 10-100-fold, about 100-1000-fold, about 1000-2000-fold, or more than about 2000-fold. In some embodiments, TILs are expanded ex vivo at least about 2-fold, at least about 4-fold, at least about 8-fold, at least about 10-fold, at least about 100-fold, at least about 1000-fold, or at least about 2000-fold.

In some embodiments, the CD3⁺ cells can be considered AML TILs due to the lack of specific biomarker. Some AML patient BM samples had low CD3⁺ TIL populations. The availability of CD3⁺ TILs can be a good prognostic marker of survival for cancer patients. High percentages of CD8⁺ TILs were also required to monitor the disease relapse. Accordingly, the present disclosure provides methods of producing at the clinical scale of, e.g., therapeutically appreciable quantity of, TILs. In specific embodiments, the method for expansion of TILs can produce 10×10⁷ TILs within 3-4 weeks after the bone marrow aspiration to meet the minimum requirement of 10⁶ TILs/kg for a 60 kg AML patient.

The complexity of AML suggests that AML patients require personalized therapies to achieve long term remission. In some embodiments, availability of CD3⁺ TILs and high percentages of CD8⁺ TILs in situ are essential in preventing disease progression or relapse, and prolonging the survival in cancer patients.

In some embodiments, TILs comprise naïve T cells, i.e., immature cells. In some embodiments, TILs are characterized by the surface expression of CD62L (L-selectin) and CCR7 (C—C Chemokine receptor type 7). In some embodiments, TILs comprise CD62L⁺ memory T cells, i.e., a subset of a naïve T cell with stemness including self-renewal and multipotent capabilities in vivo. In some embodiments, TILs isolated from bone marrow or peripheral blood samples contain CCR7⁺CD95⁻ and/or CD62L⁺CD45RA⁺ naïve T cells. In some embodiments, these cells are isolated and expanded by a modified ex vivo culture system. By using the TIL culture methods of the present disclosure, TILs can be expanded ex vivo over a three log-fold (e.g., a 1000-fold or a 2000-fold), which is useful for studying subsets of TILs and bioengineering them to fit potential clinical applications for AML. The TIL protocol of the present disclosure has incorporated co-stimulation from anti-CD3/CD28 microbeads supplemented with cytokines i.e., IL-7 and IL-15. In some embodiments, such co-stimulation increases the viability and induce expansion naïve T cells for sustainable expansion.

In some embodiments, inhibitors are added to block the PD-1, which facilitates TILs to perform potent anti-leukemic effect to AML blasts ex vivo and in vivo. In some embodiments, TILs are cultured ex vivo in the presence of a PD-1 inhibitor, e.g., an anti-PD-1 antibody, such as atezolizumab, avelumab, cemiplimab, durvalumab, nivolumab, and pembrolizumab. In some embodiments, the ratio of CD8⁺ TILs as compared to the native sample is increased in the step of culturing TILs. CD8⁺ TILs can infiltrate tumors and kill cancer cells by exerting cytotoxicity.

Bioengineering of TILs Ex Vivo

In some embodiments, the methods of the present disclosure further comprise bioengineering TILs or T cells isolated from the subject. Bioengineering can be pharmacological intervention, e.g., culturing in the presence of a PD-1 inhibitor, or genetic engineering, e.g., gene editing using a CRISPR-Cas9 system.

In some embodiments, the TILs are bioengineered to increase expression or function of a CYP27B1 gene. In some embodiments, the expression or function of the CYP27B1 gene can be increased by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) as compared to expression or function of CYP27B1 in TILs that have not been bioengineered. In some embodiments, the TILs are bioengineered to suppress expression or function of a PD-1 gene. In some embodiments, the expression or function of the PD-1 is decreased by at least 10%, at least 20%, at least 30%, at least 40%, at least 50%, at least 60%, at least 70%, at least 80%, at least 90%, or 100% as compared to expression or function of PD-1 in TILs that have not been bioengineered. Such modulation of expression or function of the CYP27B1 gene and/or a PD-1 gene can be useful for combination therapies of TIL adoptive cell therapy with PD-1 inhibitor and a hypomethylating agent (e.g., AZA).

In some embodiments, the TILs are bioengineered to improve antigen-specificity. In some embodiments, the TILs are engineered to increase expression or function of one or more of CD3, CCR7, CD62L, CD45RA, CD95, CD127, CD27, and CD28. In some embodiments, expression or function of one or more of CD3, CCR7, CD62L, CD45RA, CD95, CD127, CD27, and CD28 is increased by at least 10%, at least 20%, at least 30%, at least 40%, at least 50%, at least 60%, at least 70%, at least 80%, at least 90%, at least 100%, at least 120%, at least 140%, at least 160%, at least 180%, or at least 200% as compared to expression or function of these molecules in TILs that have not been bioengineered. Increasing expression or function of one or more of CD3, CCR7, CD62L, CD45RA, CD95, CD127, CD27, and CD28 can confer the TILs sternness and/or proliferative and functional capability as in naïve TILs.

In some embodiments, the bioengineered TILs have increased horning, proliferating, cytotoxic and/or therapeutic capabilities compared to TILs that have not been bioengineered. In some embodiments, the horning, proliferating, cytotoxic, and/or therapeutic capabilities are increased by at least 10%, at least 20%, at least 30%, at least 40%, at least 50%, at least 60%, at least 70%, at least 80%, at least 90%, at least 100%, at least 120%, at least 140%, at least 160%, at least 180%, or at least 200% in bioengineered TILs compared to TILs that have not been bioengineered.

TIL-based immunotherapy disclosed herein can be administered in combination with other therapy to treat AML in subjects. Accordingly, in some embodiments, the methods include administering autologous PD-1-inhibited-TILs in combination with AZA and PD-1 inhibitors as a viable combination therapy to treat AML and prevent relapse. Autologous PD-1-inhibited-TILs in combination with AZA generate a potent anti-leukemic effect and overcome the immune-escape of leukemia blasts by reducing the amount of LSCs and AML blasts thereby extending the survival rate of AML subjects. Additionally, specificity of using the same patient's cells will minimize the likelihood of unwanted side effects of combination treatment for elderly AML patients in the clinic. Accordingly, in some embodiments, the methods include administering a therapeutically effective amount of a PD-1 inhibitor, e.g., atezolizumab, avelumab, cemiplimab, durvalumab, nivolumab, and pembrolizumab, and a hypomethylating agent, e.g., 5-Azacytidine (AZA).

### EXAMPLES

The following examples are offered by way of illustration and not by way of limitation.

### Example 1: Isolation of TILs from AML Patient Bone Marrow Samples

In an example, human CD8⁺CD3⁺ cytotoxic T effector cells were enriched from AML BM and their PD-1 expression was inhibited for generating PD-1-inhibited-TILs. This method generated PD-1-inhibited-TILs in sufficient amounts and in a quicker time frame. Separation of CD3⁺ T cells from bone marrow mononuclear cells (BM-MNC) was performed by using CD3 microbeads (Cat. #130-050-101, Miltenyi Biotech, Germany) and a MiniMACS™ Separator with an MS Column according to the manufacturer's protocol. Selected CD3⁺ T cells were considered AML TILs.

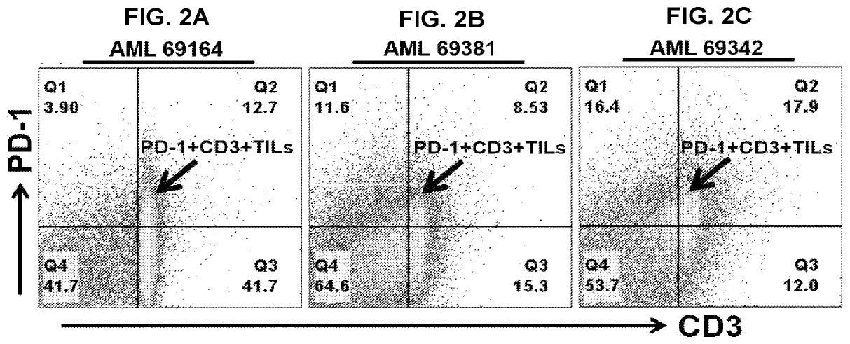

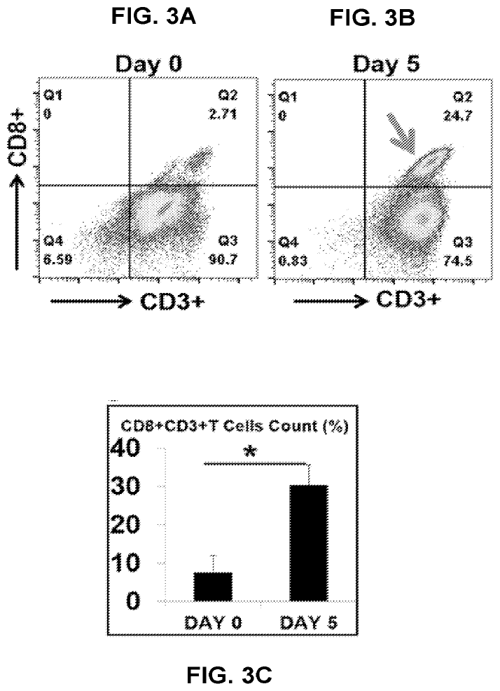

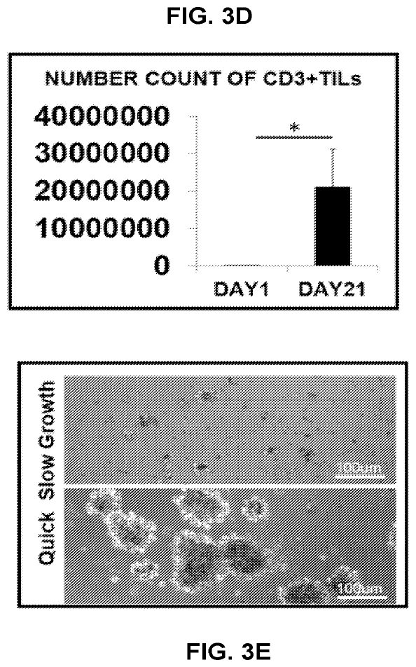

Following ex-vivo expansion of low number TILs using media with cytokines, AML patient BM samples were screened and CD3⁺ T cells were present in some samples (N=8, FIGS. 2A-2C). As shown in FIGS. 3A-3C), both CD3⁺ and CD8⁺ TILs could be expanded quickly. CD3 microbeads were applied to pull down the CD3⁺ TILs according to the manufacturer's protocol (Miltenyi Biotech, Germany). The non-CD3⁺ cells (feeder cells) were pre-treated with 10 μg/mL mitomycin-C for 2 hours to cease proliferation. CD3⁺ TILs and feeder cells were co-cultured in RPMI-1640 supplemented with Interleukin-2 (IL-2). Media change was performed every 2 to 3 days. TILs were stimulated with 30 ng/mL human anti-CD3 antibodies (OKT3, Ortho Pharmaceutical, Raritan, NJ). On days 5-7, cultures were counted and split as necessary to T25 or T75 flasks for expansion before the next engineering. Since there are also a group of patients with low ratio of TILs (data not shown), to apply the same TIL therapy for these AML patients, a TIL culture method was developed to expand TILs from BM with extremely low CD3⁺ T cells. A low number of TILs ex vivo can be cultured to a clinical application scale, potentially leading to over a thousand-fold increase in TILs from about 20,000 CD3⁺ T cells (1 ml BM aspirate) to over 30,000,000 CD3⁺ T cells during the period of 3-4 weeks (N=10, FIG. 3D). Phenotypic characterization of ex vivo expanded TILs can be examined with biomarkers of CD3, CD4, CD8, CD56, CD25, CD137, CCR7, CD45RA, CD45RO, CD62L, CD95, CD127, CD132, TCF-1, TOX, PD-1, CTLA-4, etc. Data will be acquired on an LSRII flow cytometer (FACS, BD Biosciences) and analyzed using FlowJo software. FIG. 3D is a graphical representation of the number of CD3⁺ cells at Day 1 and Day 21. FIG. 3E presents representative images of 2 AML patients' TIL culture; *P<0.05.

Sufficiently large amounts of CD3⁺ TILs were isolated and enriched ex vivo to serve as alternative candidate cell vehicles for autologous treatment for AML. PD-1-inhibited-TILs were generated by treating ex vivo expanded TILs with PD-1 inhibitors. There were PD1⁺CD3⁺T cells inside the AML BM. First, AML patient BM samples were screened for CD3⁺T cells (FIGS. 2A-2C). Further analyses revealed that 39.54% CD3⁺ T cells were PD-1⁺ (arrows, FIGS. 2A-2C). FIGS. 2A-2C are density plots from FACS analysis for the expressions of PD-1 and cluster of differentiation protein 3 (CD3). PD-1⁺CD3⁺ TILs are likely to have lost anti-leukemic activity against AML blasts which are reported to constitutively express high titers of PD-L1 to inhibit T-cell anti-tumor responses.

Conventionally, T-cell culture protocols are experimentally established in a hierarchy through proliferation of CD3⁺T cells, differentiation into CD8⁺ T cells, and functional assay. Disclosed here are efficient methods of isolating and expanding CD3⁺ TILs ex vivo, thus isolating and expanding BM TILs. The AML patient shown in FIG. 3A was found to have CD3⁺TILs in the BM (FIG. 3A). FIGS. 3A-3B are density plots from FACS analysis for the expressions of CD8 and CD3 in TILs in culture. CD3 microbeads were applied to pull down the CD3⁺ TILs which were then co-cultured with supporting feeder cells in RPMI-1640 with IL-2. On day 5, the cells were collected and stained for flow cytometry (FACS) analyses to compare TILs percentages before and after short-term cell cultures. The red arrow indicated an 11-fold increased population of CD8⁺CD3⁺ T cells after the 5-day culture (FIG. 3B). Also, there were significantly increased CD3⁺ TILs populations in our T cell culture system (from 44.65% of Day 0 to 96.4% of Day 5, FIG. 3C). FIG. 3C is a graphical representation of the percentage of cells that express CD8 and CD3 at Day 0 and Day 5.

### Example 2: Anti-Leukemic Effect of TILs in MOLM-14 Ex Vivo

The function of ex vivo expanded TILs was examined. Patient-derived CD3⁺TILs were co-cultured with MOLM-14 cells. MOLM-14 is a human AML cell line with the FLT3 ITD mutation. CFSE was used to label the MOLM-14 blasts. After a 4-hour co-culture, the cells were collected and stained for FACS analyses to examine the anti-leukemia response. A clear population of dead cells was observed within the CFSE⁺MOLM-14 population (0.06% to 0.4%). The quantitative data suggested a significant difference between the TIL treated and non-treated experimental groups. The efficacy of TIL treatment was also observed. About 58.7-82% of primary AML CD117⁺ cells were PD-L1⁺ (n=3). In addition, expanded CD3⁺ TILs were found to have a large fraction of PD-1⁺ cells. MOLM-14-derived PD-L1 ligands might protect blasts through immune-escape by blocking the function of PD1⁺TILs, as other cancer cells did. Nivolumab, a PD-1 inhibitor, was applied to treat expanded primary TILs.

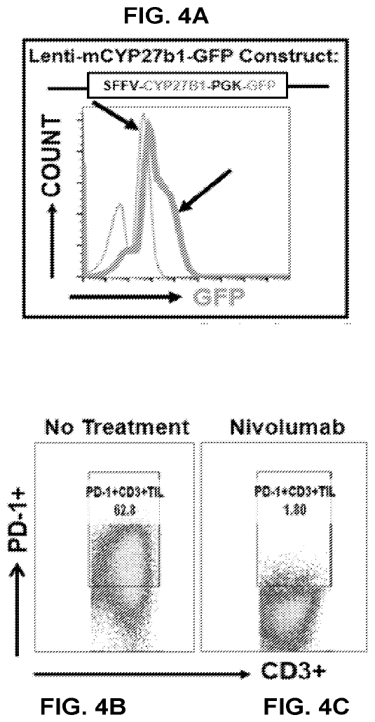

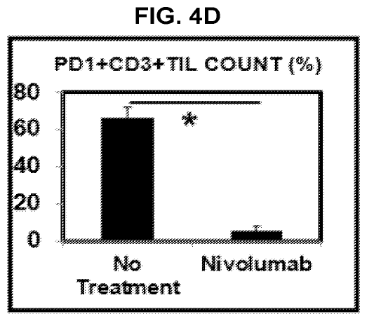

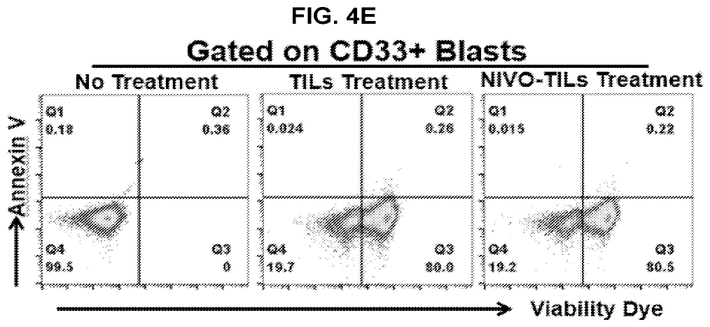

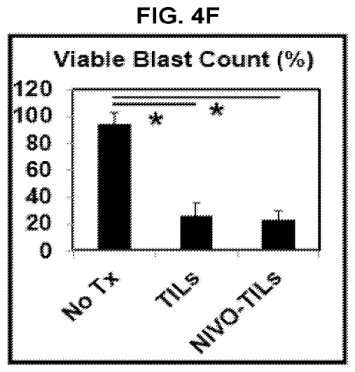

Vitamin D is known to play essential roles in bone metabolism and calcium homeostasis, and recognized to be involved in new immune-modulatory roles and cancer treatment through the extra-renal synthesis. A gene therapy system was developed using CYP27B1, encoding an enzyme of producing active 1.25(OH)2D3, combined with 5-Azacitidine (5-AZA, a hypomethylating agent) to improve clinical symptoms and prolong the survival of AML mice without hypercalcemia. Vitamin D combined with 5-AZA can not only reduce LSCs' frequency, but also maintain the stemness of human Mesenchymal Stem Cells (MSCs) and HSCs for regenerating healthy microenvironment and hematopoiesis. TILs consisting of CD3⁺T-cells were present in some AML patients, and they were isolated, quickly expanded, pharmaceutically and genetically bioengineered to perform cytotoxic functions ex vivo (N=8 among 11 samples). FIGS. 4A-4F provide characterization of the bioengineer-expanded TILs lentivirally and pharmaceutically ex vivo. Those ex vivo expanded TILs were genetically engineered by the lentiviral system to express the stable ectopic gene CYP27B1, encoding an enzyme responsible for the generation of active Vitamin D, known to suppress macrophages, the key mediator for CRS. The lentivirus is a cost-effective way to integrate exogenous genes into a host cell genome with long-term stable expression and its latest 3rd generation system has been established by the inventors. FIG. 4A is a FACS plot of GFP expression after lentiviral transduction into TILs. FIGS. 4B and 4C are FACS plots of PD-1 expression in CD3⁺TILs with or without treatment of an inhibitor of PD-1 expression, Nivolumab. FIG. 4D is a graphical representation of the percentage of CD3⁺TILs that express PD-1 with or without treatment using Nivolumab. FIG. 4E is a FACS plot to measure cytotoxicity following no treatment, treatment with CD3⁺TILs with or without exposure to Nivolumab. FIG. 4F is a graphical representation of the percentage of viable blast count in the cytotoxic tests. N=3. CD3⁺ TILs have been engineered to be CYP27B1⁺GFP⁺TILs (FIG. 4A). The construct CYP27B1 is functional both in vitro and in vivo. Therefore, similar approach will be used to generate CYP27B1⁺ TILs ex vivo.

Blocking PD-1 was very effective, a significant reduction of PD-1⁺CD3⁺ TILs from 62.8% to 1.8% (FIG. 4B, 4C). The cytotoxicity will significantly improve when TILs with PD-1 inhibition are used to treat blasts from the same patient. These PD-1⁺CD3⁺ TILs would have lost anti-leukemic activity against AML blasts which are reported to constitutively express high titers of PD-L1 to inhibit T-cell anti-tumor responses. The PD-1 expression of ex vivo expanded TILs was almost entirely blocked by Nivolumab, an anti-PD-1 monoclonal antibody (FIG. 4B-4D). Cytotoxic effects of TILs on autologous CD33⁺ AML Blasts were clearly observed (N=3, FIG. 4E, 4F).

### Example 3: Anti-Leukemic Effect of TILs in Autologous AML Blasts Ex Vivo

Expanded TILs were evaluated for their ability to kill same patient derived AML blasts ex vivo Killing tests were performed by co-culturing Programmed-TILs with primary AML blasts from the same patient (isolated by CD33-microbeads pull down). About 1×10⁵ primary AML blasts per well (24-well-plate) are treated for different time points including 4 hours and 16 hours before analyzing cell viability and programmed cell death by FACS. The ratio of TILs to AML blasts will be in the range of Five experimental groups are evaluated to test the anti-leukemic effects of Programmed-TILs: (1) Group 1: Primary AML blasts with no treatment; (2) Group 2: Primary AML blasts treated with TILs (ratio: 10:1); (3) Group 3: Primary AML blasts treated with CYP27B1+TILs (ratio: 10:1); (4) Group 4: Primary AML blasts treated with Nivo-TILs (Nivolumab-treated; ratio: 10:1); (5) Group 5: Primary AML blasts treated with Nivo-CYP27B1⁺TILs (ratio: 10:1). After the treatment, the cells are collected and stained for FACS analyses according to manufacturers' protocols to examine the anti-leukemic effect of bioengineered-TILs. For cytotoxic studies, the cell viability-dye, and Annexin V are applied. Analyses and graphs are generated using the GraphPad Prism software to evaluate significance.

### Example 4: AML PDX Models

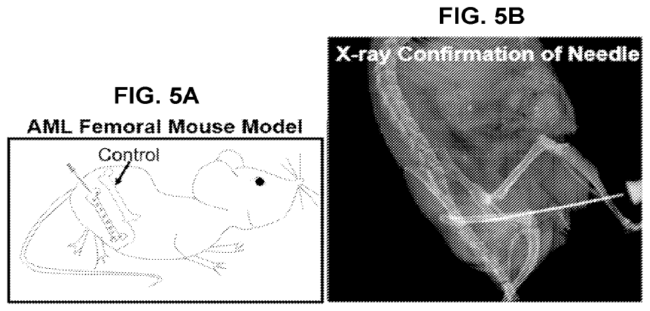

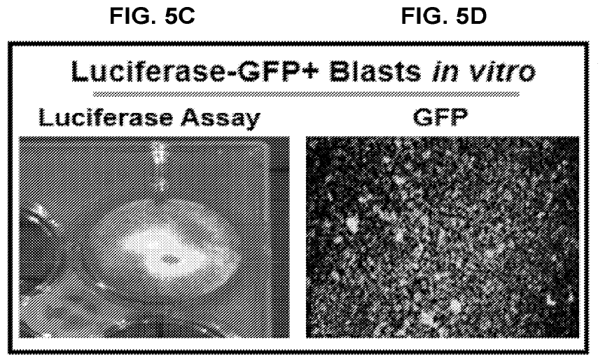

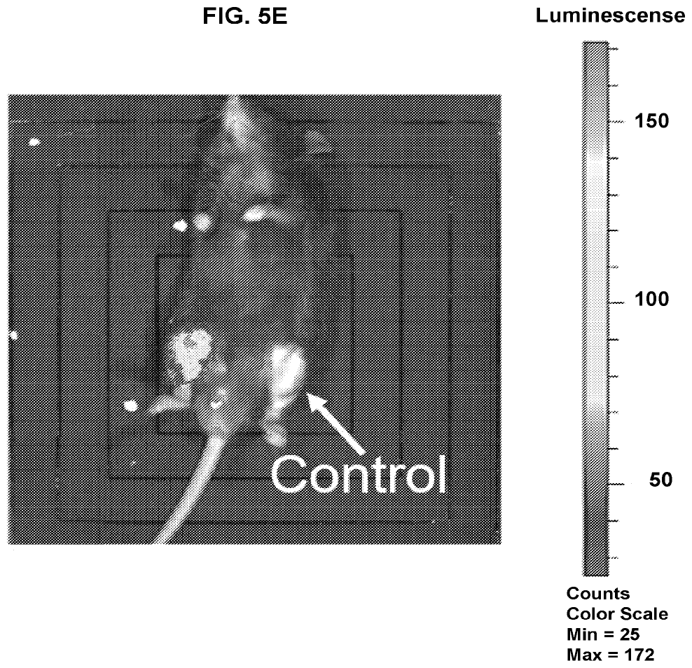

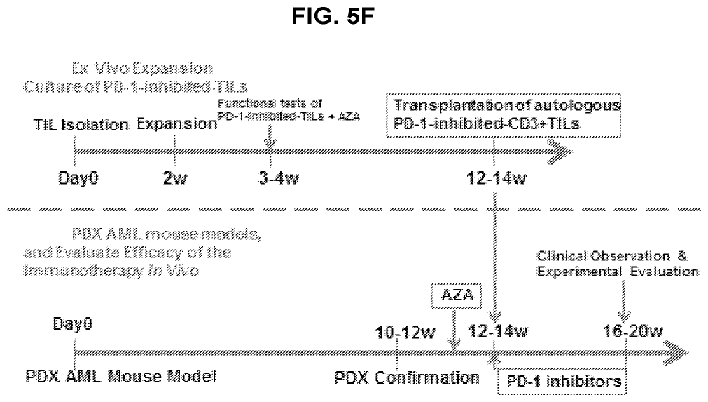

ML PDX models have been developed to investigate therapies in immune-deficient mice (NSG or NRG). Previously, an AML xenograft mouse model was established by transplanting human AML cell line MOLM-14 cells into NRG mice. To determine whether ex vivo expanded TILs still maintain their Ag-specific, proliferation and cytotoxic capability in vivo, a localized AML PDX mouse model was generated to create an AML or non-AML microenvironment to illustrate the function of bioengineered-TILs. The AML PDX mouse model was generated by intrafemoral transplantation of AML cells. FIGS. 5A-5F are directed to the generation of femoral PDX AML mouse model. FIG. 5A is an illustration of the femoral PDX AML mouse model. FIG. 5B is an X-ray image of injection. FIGS. 5C, 5D, and 5E are bioluminescence images of Luciferase-GFP⁺ AML blasts in vitro and in vivo inside the bone marrow of AML mice, respectively. The luciferase & fluorescent reporter double-labeled MOLM-14 AML cells were generated in vitro (FIG. 5C, 5D) and then injected (1×10⁵) into the B6 mouse femur to create AML or non-AML microenvironment (control). The MOLM-14 cells were observed live by Bioluminescence imaging and GFP histology (FIG. 5E). Similar approaches are applied to the AML PDX model by injecting 1×10⁶ fluorescent reporter-labelled AML PDX cells into the femur of NRG mice. The progression of disease is monitored by luciferase imaging and some mice are euthanized for confirmation 2 weeks after injection. Femoral BM, splenocytes, and peripheral blood (PB) are harvested and assessed by FACS analyzing percentage of hCD33, hCD34, hCD38, hCD117, hCD13, etc. for AML LCSs/blasts.

To generate AML PDX mice, adult NRG mice (8-10 weeks old) are sub-lethally irradiated with 250 cGy of total body irradiation 24 hours before tail vein injection of T cell depleted AML cells (5×10⁶ per mouse). Daily monitoring of mice for symptoms of disease (ruffled coat, hunched back, weakness, motility) are performed. Mice are euthanized 10-12 weeks after AML injection. Femoral BM, splenocytes and peripheral blood (PB) are harvested. Human AML engraftment is assessed by FACS analyzing percentage of human (h) CD45⁺ CD33⁺ cells. In addition, percentage or absolute number of AML cells positive for hCD34, hCD38, hCD117, hCD13 and etc. is determined at time of euthanasia respectively. Primary AML cells from either fresh samples or cryopreserved samples are obtained with informed consent and IRB approval from LLU. FIG. 5F schematically depicts the protocol for generating PDX AML mouse models and evaluating efficacy of the immunotherapy in vivo.

### Example 5: Evaluation of Therapeutic Effect of Autologous TILs and Chemotherapy in AML PDX Mice In Vivo

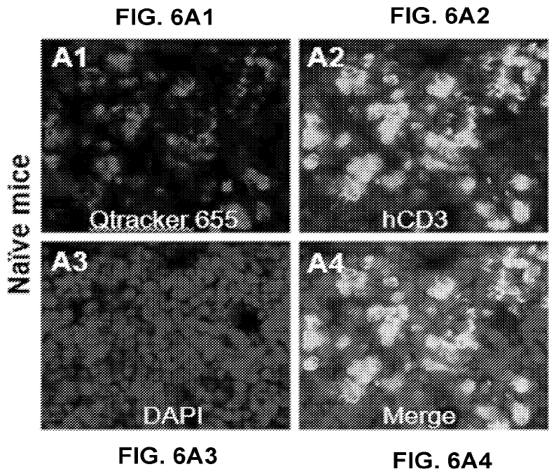

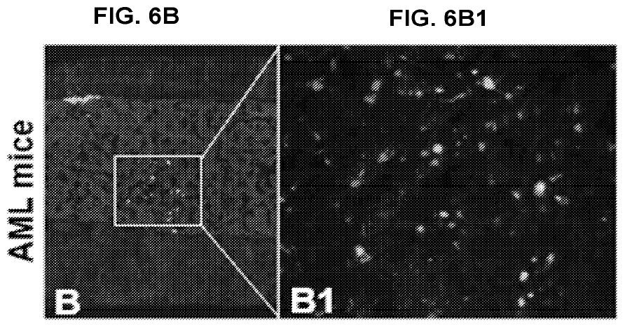

The therapeutic effect of autologous bioengineered-TILs in combination with Nivolumab on leukemic cell burden and survival of AML PDX mice are evaluated in vivo. In preliminary studies (FIG. 6), to determine whether ex vivo expanded TILs would go back to BM, Nivo-TILs were transplanted into NRG mice through tail IV injection. Many engrafted TILs were found proliferating inside BM and being CD3⁺ T cells by immunohistochemistry 2 weeks after transplantation. However, these Nivo-TILs were found to be PD-1⁺ (data not shown), suggesting engrafted TILs might be affected by their surrounding environment and the persistent requirement of PD-1 inhibitors for TILs after the transplantation. FIGS. 6A1-6A4 and FIGS. 6B1-6B2 are photographic images of femoral bone marrow in naïve environment and when transplanted PD-1-inhibited-TILs, respectively.

### Example 6: Effect of AZA and 1.25(OH)2D3 on AML Cells

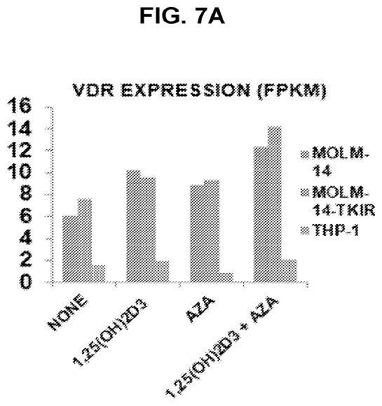

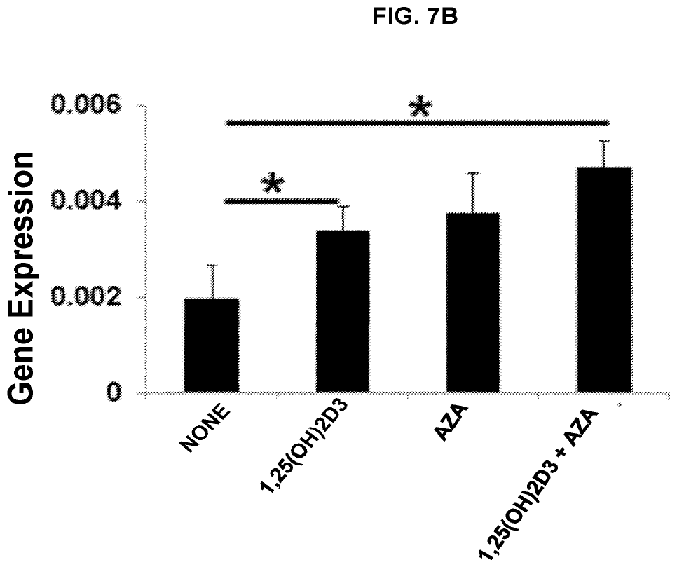

Experiments are done to determine whether the regaining of CYP27B1 and newly synthesized Vitamin D rescue bone structures in AML BM. Bone structures in AML BM and non-AML BM before and after treatment were evaluated by Micro-computed Tomography (μCT) for bone volume, bone mineral density, connectivity density and spaces. To understand the mechanism of 1.25(OH)₂D₃ based treatment for AML, in context of combination with AZA, the RNA-Seq transcriptome profiling of different AML cells lines were performed, which were pre-treated with either 1.25(OH)₂D₃ alone, AZA alone or their combination in vitro for 48 h. FIGS. 7A-7B are graphical representations of RNA-seq analysis of AML cells treated with single agents-1.25(OH)2D3 and AZA and their combination. FIG. 7A is a graphical representation of expression of human VDR and FIG. 7B is a graphical representation of VDR expression in cells treated with single agents-1.25(OH)2D3 and AZA and their combination as confirmed by quantitative PCR (qPCR) experiments. Protein change of Nestin, mCD90, mCD73, mCD11b, mCD11c, RUNX2, BMP2, BMP9, Osterix, DLX5, Osteocalcin, RANK, MMP9, etc. can be measured for AML-related BM changes. Autologous PD-1-inhibited-TILs in combination with AZA generate a potent anti-leukemic effect and overcome the immune-escape of leukemia blasts by reducing the amount of LSCs and AML blasts thereby extending the survival rate of AML subjects. Additionally, specificity of using the same patient's cells will minimize the likelihood of unwanted side effects of combination treatment for elderly AML patients in the clinic.

### Example 7: Confirmation of the Presence of TILs in Bone Marrows of AML Patients

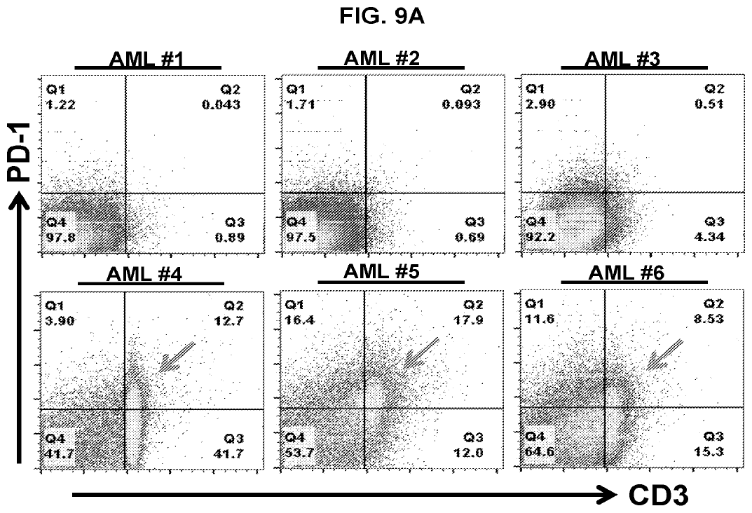

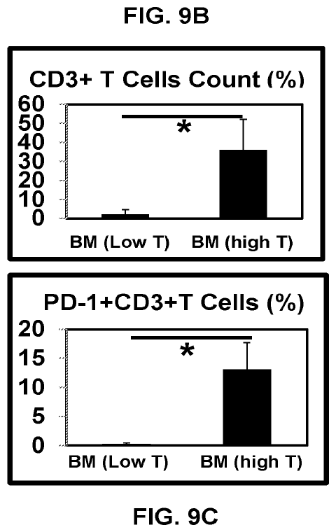

Autologous TILs based therapies could be a novel therapeutic strategy for AML if the following is done: 1) phenotypically identify TILs, 2) expand TILs ex vivo to sufficient numbers for clinical use, 3) demonstrate cytotoxic effect to autologous AML blasts and 4) bioengineer TILs to restore their Ag-specific cytotoxic functions. To this end, AML patient BMMNC (Patients #1-10, Table 1) were screened. A different degree of CD3⁺ T cells infiltration could be detected in all the tested samples. FIGS. 9A-9C depict immunophenotyping of TILs from AML patients' bone marrow (BM) samples. FIG. 9A shows density plots from FACS analysis for expression of PD-1 and CD3. FIGS. 9B and 9C are graphical representations of the percentages of CD3⁺ T cells and PD-1⁺ CD3⁺ T cells in two groups of patients with low and high numbers of CD3⁺ T cells or PD-1⁺ CD3⁺ T cells, respectively. Overall, two groups of patients with low (“Low”: upper FACS plots, FIG. 9A; Patient #1-3, 7, Table 1) and high (“High”: lower FACS plots, FIG. 9A; Patient #4-6, 8-10, Table 1) numbers of CD3⁺ TILs (2.3% vs 32.6%, respectively, P<0.05) (FIG. 9B). This finding is interesting in that certain AML blast environment elicit a suppressed T cell response. Further analyses revealed that 13% of CD3⁺ T cells were PD-1⁺ (arrows, FIG. 9A, C). These PD-1⁺ TILs are likely to have lost anti-leukemic activity against AML blasts, which could be restored functionally by using PD-1 inhibitors.

### Example 8: Ex Vivo Expansion of TILs from AML BMs Using a Modified Protocol

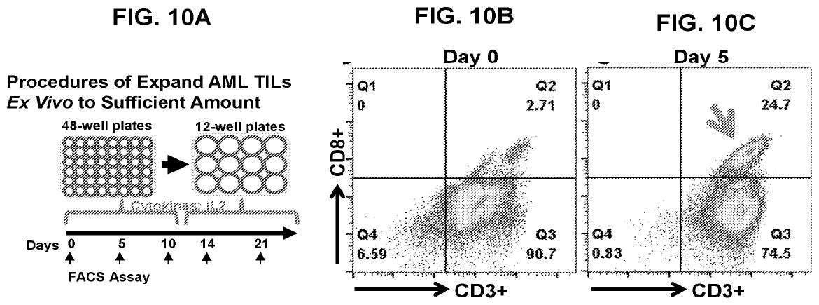

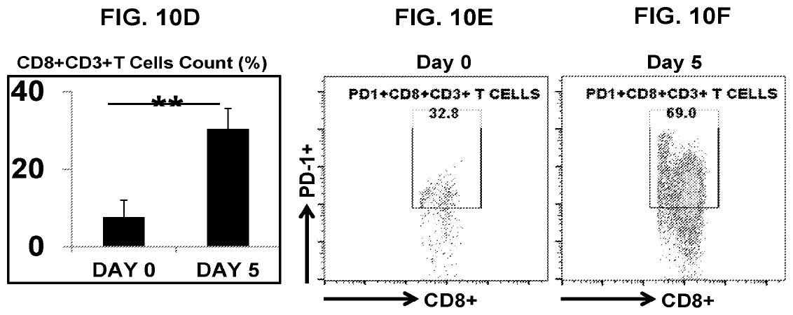

The ex vivo expandability of AML TILs was examined using the T cell culture system of the present disclosure (FIG. 10A). FIGS. 10A-10F depict expansion of high number CD3⁺TILs ex vivo. FIG. 10A is an illustration of the experimental procedures of ex vivo culture of isolated CD3⁺TILs. FIGS. 10B and 10C are representative FACS plots showing the percentage data of CD3⁺ or CD8⁺ cells on day 0 and day 5. FIG. 10D is a graphical representation of the cumulative FACS percentage data of CD8⁺CD3⁺ T cells on day 0 and day 5. FIGS. 10E and 10F are representative FACS plots showing the PD1⁺ CD8⁺ cells on day 0 and day 5. Where applicable, data are means±SEM and were analyzed by Student t-test. ** P<0.01.

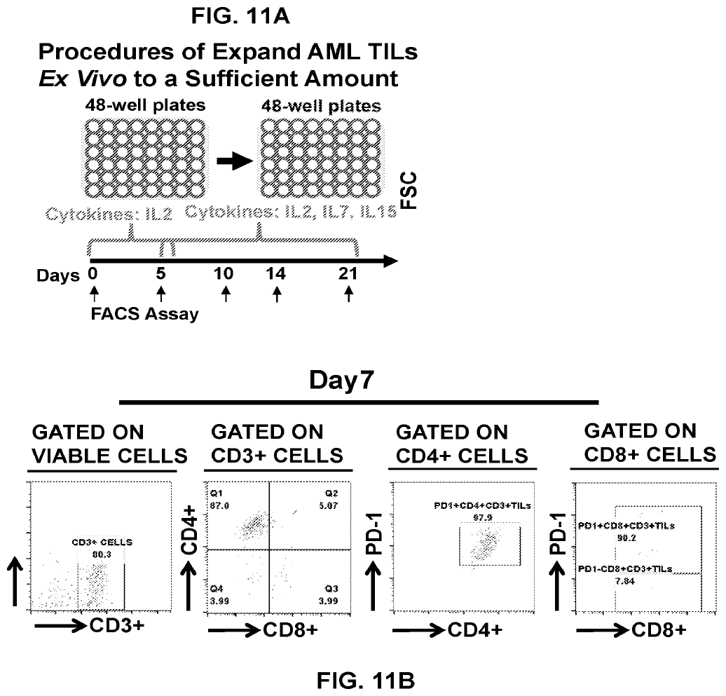

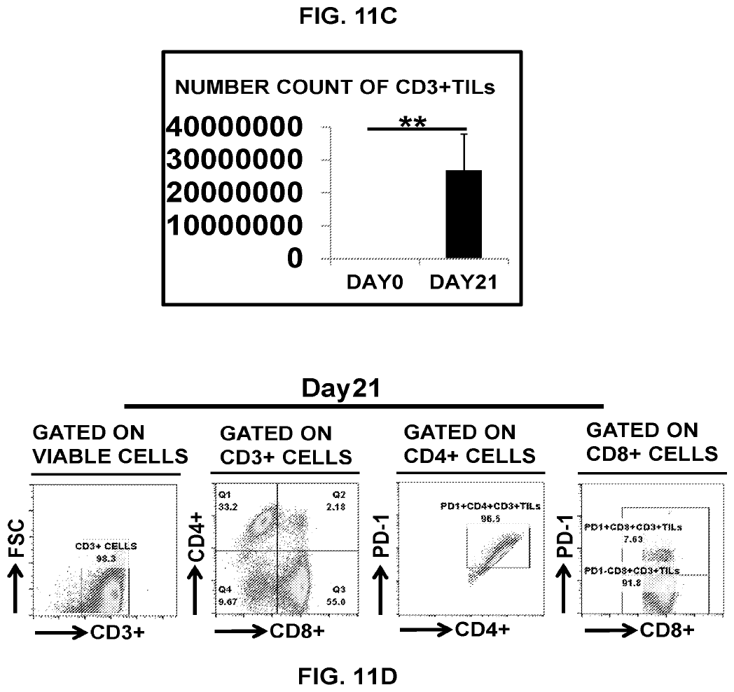

From the “High” group, 0.5 to 2×10⁶ CD3⁺ T cells/ml were obtained using CD3 microbeads. After magnetic separation, these cells (Patient #10, Table 1) were cultured with supporting feeder cells in RPMI-1640 supplemented with IL-2. A 4-fold increase of the CD8⁺CD3⁺ T cell population (red arrow) was obtained after the 5-day culture (30.4% on Day 5 vs 7.6% on Day 0, P<0.01, FIG. 10B-10D). In contrast, from the “Low” it was found challenging to obtain a sufficient number of TILs. Thus, to expand these cells a modified ex vivo culture protocol was utilized (FIG. 11A). Ten vials of AML BMMNC with low T cell numbers were used in this experiment (Patients #11-20, Table 1). CD3 microbeads were applied to pull down the TILs from 1 ml of each BMMNC sample; 2 to 5×10⁴/ml CD3⁺ T cells were obtained and cultured with CD3/CD28 microbeads without feeder cells in RPMI-1640 supplementing them sequentially with IL-2, IL-7, and IL-15 (see experimental methods for details). At different time points, cells were collected and stained for FACS analyses to determine their immunophenotypes. At the early stage (day 7), most CD3⁺ TILs were found to be CD4⁺ (87%, FIG. 11B), while also expressing PD-1⁺ (97.9%). At day 21, a three-log increase of CD3⁺ TIL populations was observed (26795470 on day 21 vs 25600 on day 0, P<0.01, FIG. 11C). Also at day 21, the percentage of CD4⁺ reduced to 33.2%, while CD8⁺ TILs increased to 55% with low expression of PD-1 (7.63%, FIG. 11D). FIGS. 11A-11D depict expansion of low number CD3⁺TILs ex vivo. FIG. 11A is an illustration of the experimental procedures of ex vivo culture of isolated CD3⁺ TILs. FIG. 11B is a representative FACS plots showing the CD4⁺ CD3⁺ or CD8⁺ CD3⁺ T cell subsets and their PD-1 expression on day 7. FIG. 11C is a graphical representation of the cumulative FACS percentage data of CD3⁺ T cells on day 21. FIG. 11D are representative FACS plots showing the CD4⁺ CD3⁺ or CD8⁺ CD3⁺ T cell subsets and their PD-1 expression on day 21. Where applicable, data are means±SEM and were analyzed by Student t-test. **P<0.01.

### Example 9: Bioengineering Expanded TILs Pharmaceutically and Genetically Ex Vivo

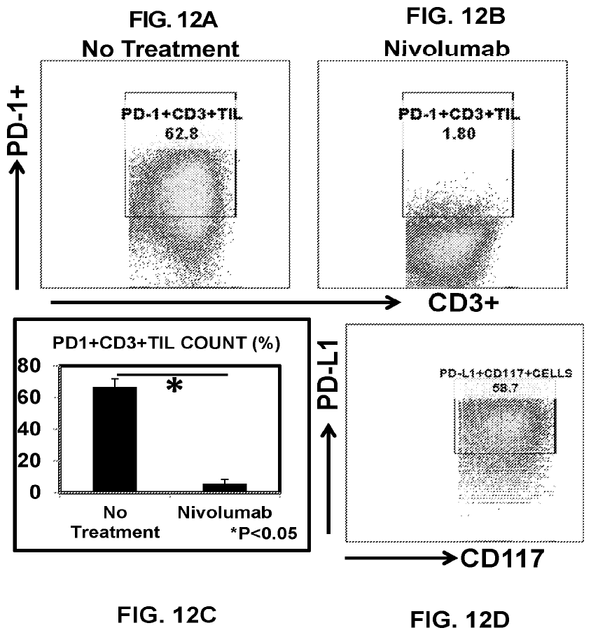

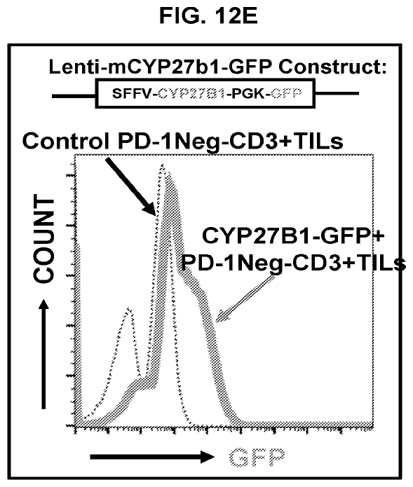

The possibility of pharmaceutically and genetically bioengineering expanded TILs ex vivo were investigated (FIG. 12). The PD-1 pathway has received considerable attention because of its negative role during acute T cell activation and being a marker for T cell exhaustion. Nivolumab, an FDA-approved monoclonal antibody PD-1 inhibitor, was used to suppress PD-1 expression on TILs, as evidenced by FACS analyses (significantly reduced from 62.8% to 1.8%, FIGS. 12A-12C). Previously, a new Vitamin D gene therapy was reported to treat AML by overexpressing the CYP27B1 ectopic gene, which encodes the 1-alpha-hydroxylase to generate active Vitamin D in situ. In this study, using a lentivirus system, ex vivo expanded TILs were also genetically engineered, which were demonstrated to overexpress the CYP27B1 ectopic gene (arrow, FIG. 12E). The anti-leukemia function of CYP27B1⁺ TILs was examined and whether TILs are a potential cell vehicle candidate for gene therapies was examined FIG. 12A-12E depict bioengineering of primary AML TILs ex vivo. FIGS. 12A and 12B are representative FACS plots showing the percentage data of PD-1 expression in CD3⁺ TILs with or without treatment of Nivolumab. FIG. 12C is a graphical representation of the cumulative FACS percentage data of PD-1 expression in CD3⁺ TILs with or without treatment of Nivolumab; N=3. FIG. 12D is a representative FACS plot of PD-L1 expression in CD117⁺ AML blasts. FIG. 12E is a graphical representation of the FACS analyses of GFP expression in PD-1Negative-CYP27B1⁺TILs after lentiviral transduction. Where applicable, data are means±SEM and were analyzed by Student t-test. * P<0.05.

### Example 10: Difficulties with Expansion of CD3⁺ TILs in Some AML Patients

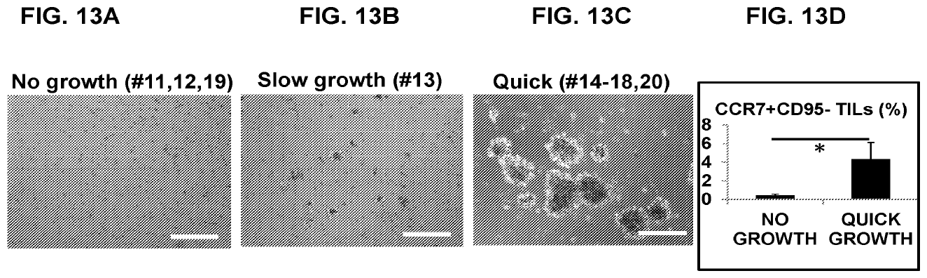

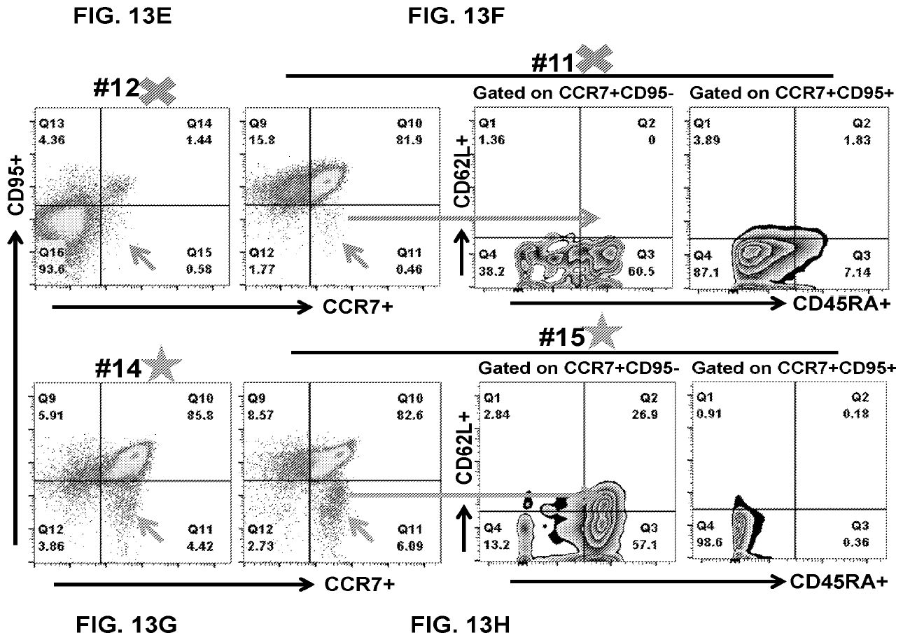

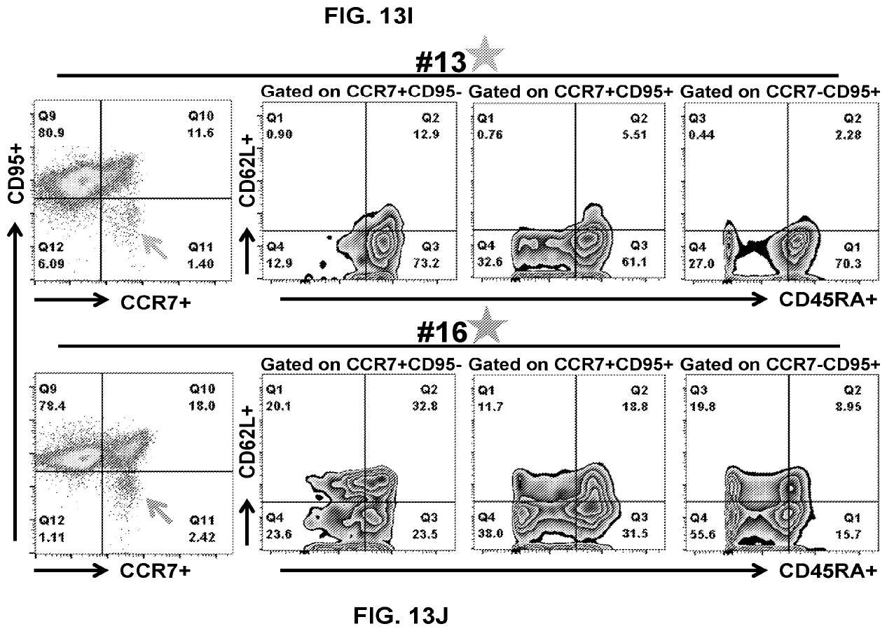

During the culture of TILs from 10 AML patient samples, one consistent aspect for TIL cultures (n=10) was that the proliferation status of TILs during the early stages (days 3-5) was a good predictor for whether TILs (representative images of early TIL clusters, FIG. 13C) could be expanded to clinical scale in later stages. It was found that not every sample could generate TILs ex vivo (FIG. 13). After CD3 microbead pull-down, CD3⁺ T cells were present in these BMMNC samples (patients #11, 12, 19), but they failed to expand (FIG. 13A) and generate TIL clusters (FIG. 13C). There was another sample (patient #13) which could generate small clusters (FIG. 13B), but it grew relatively slow compared to those quickly expanding TILs (patients #14-18, 20, FIG. 13C).

FIGS. 13A-13J depict correlation of the existence of naïve TILs in AML patients' BM samples and their proliferation capabilities ex vivo. FIGS. 13A, 13E, and 13F represent “no growth” TILs (from patients #11, 12, 19). FIGS. 13B and 13I represent “slow growth” TILs (from patient #13). FIGS. 13C, 13G, 13H, and 13J represent “quick growth” TILs (from patients #14-18, 20). FIGS. 13A-13C are representative phase-bright images showing different growth of CD3⁺ TILs; Scale bar: 100 μm. FIG. 13D is a graphical representation of the cumulative FACS percentage data of CCR7⁺ CD95− TILs between No growth and Quick growth. FIG. 13E shows a representative FACS plots of TILs with no growth from Patient #12. Red arrow indicates CCR7⁺ CD95− cells. FIG. 13F shows a representative FACS plots of TILs with no growth from Patient #11. Short red arrow indicates CCR7⁺ CD95⁻ cells; Long red arrow indicates CCR7⁺ CD95⁻ CD62L⁺CD45RA⁺ cells. FIG. 13G shows representative FACS plots of TILs with quick growth from Patient #14; short green arrow indicates CCR7⁺ CD95− cells. FIG. 13H shows representative FACS plots of TILs with quick growth from Patient #15. Short green arrow indicates CCR7⁺ CD95⁻ cells. Long green arrow indicates CCR7⁺ CD95⁻ CD62L⁺CD45RA⁺ cells. FIG. 13I shows representative FACS plot of TILs with slow growth from Patient #13; Brown arrow indicates CCR7⁺ CD95⁻ cells. FIG. 13J shows representative FACS plot of TILs with quick growth from Patient #16; Green arrow indicates CCR7⁺CD95⁻ cells. In each of the foregoing figures, # indicates Patient No; Red X mark indicates no growth; Brown Star mark indicates slow growth; Green Star Mark indicates quick growth.

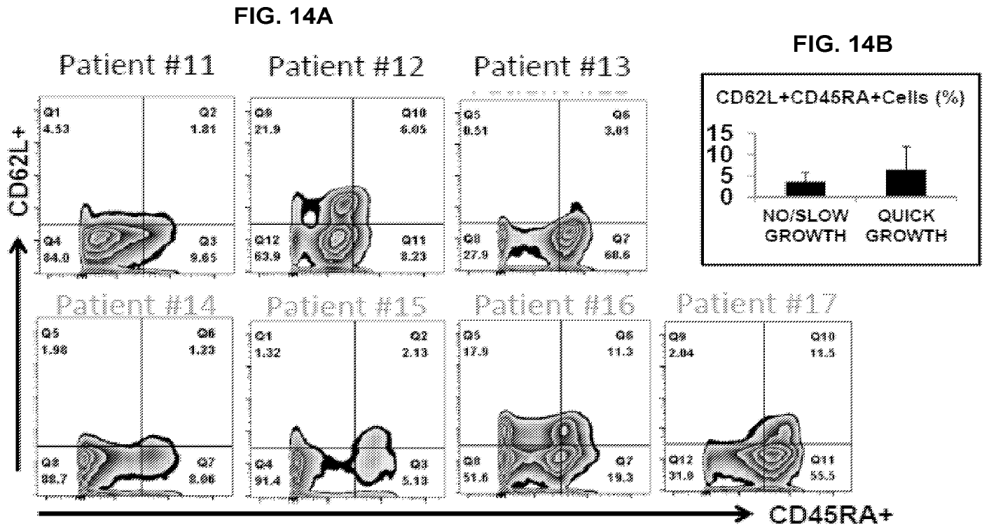

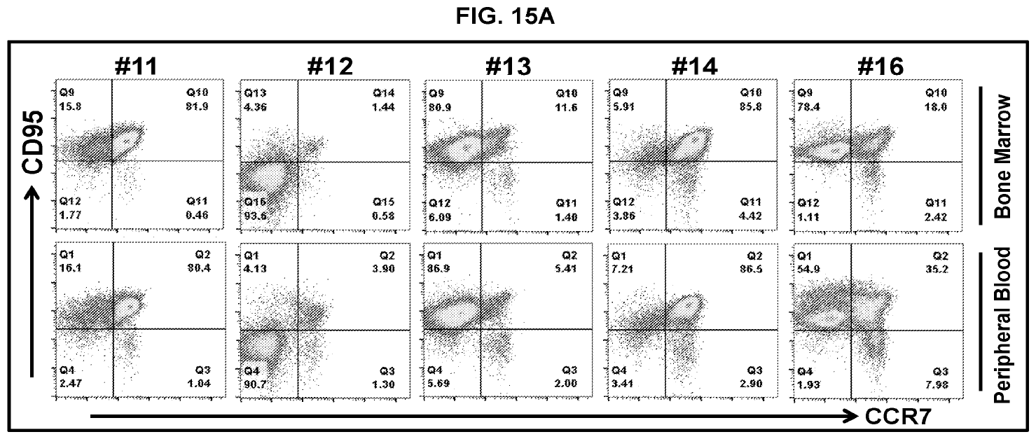

To investigate the mechanism underlying differential growth capabilities of TILs, an immunophenotypic comparison of these AML BMMNC by using biomarkers for naïve T cells, including CD62L, CD45RA, CCR7, CD95, were performed. No significant difference of CD62L⁺CD45RA⁺ naïve TILs was found between the no/slow growth BMMNC and the quick growth BMMNC (P=0.38, FIG. 14). FIGS. 14A-14B depict comparison of CD62L⁺CD95⁺ TILs in the AML Patients' bone marrow mononuclear cells (BM-MNC). FIG. 14A shows representative FACS plots showing the percentage data of CD62L⁺CD45RA⁺ TILs from different AML patient BMMNC; Red patient numbers: no growth; Brown patient numbers: slow growth; Green patient numbers: quick growth. FIG. 14B is a graphical representation of the cumulative FACS percentage data of CD62L⁺CD45RA⁺TILs between No/Slow growth and Quick growth. Where applicable, data are means±SEM and were analyzed by Student t-test.

However, there was a significant loss of CCR7⁺CD95− naïve T cell population (red arrow, FIG. 13E, 13F) in patients #11 and #12 (9.6-fold decrease, 0.45% of no growth vs 4.31% of quick growth, P<0.05, FIG. 13D). There were clear CCR7⁺CD95− naïve T cell populations in patients #14, #15, and #16 (green arrow, FIG. 13G, 13H, 13J), part of which also expressed CD62L⁺CD45RA⁺ naïve biomarkers. When comparing the detailed immunophenotypic pattern of patient #13 (slow growth) with patient #16 (quick growth), there was a 3-fold increase of CD62L⁺CD45RA⁺ cells in patient #16 BMMNC versus patient #13 BMMNC in each compartment of CCR7⁺ or CD95⁺ subpopulations (FIGS. 3I, 3J).

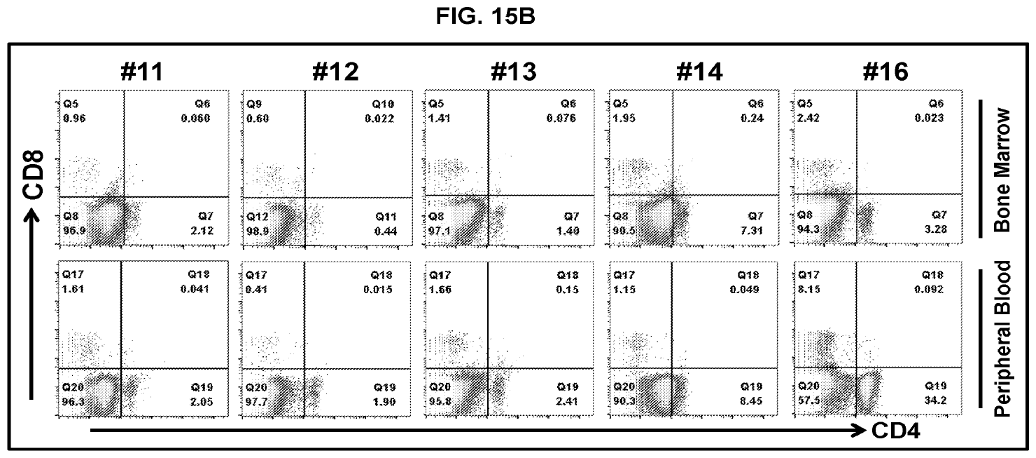

The data suggest that to effectively expand TILs to a sufficient amount, the CCR7⁺CD95− naïve T cell population in AML patient BM are needed to support the quick expansion ex vivo. Alternative sources of T cells for TILs therapy in patients with low BM TILs were developed, including peripherally isolated T cells expanded by the methods disclosed herein. Similar patterns of naïve T cells and differentiated T cells were identified in the peripheral blood (PB) and BM samples of same patients (FIGS. 15A, 15B). Next, expansion culture experiments revealed similar growth patterns between PB and BM samples including no expansion of #11 and #12 PB samples ex vivo (data not shown). FIGS. 15A-15B depict comparison of naive T cells and differentiated T cells in bone marrow and peripheral blood of same patients (#11-#16). FIG. 15A shows representative FACS plots of naïve T cells for CCR7 and CD95 expression. FIG. 15B shows representative FACS plots of differentiated T cells for CD4 and CD8 expression. In all, the presence of naïve T cells in the marrow of a subgroup of AML patients was found to be critical for sufficient expansion of TILs and further treatment development.

### Example 11: Functional Characterization of Ex Vivo Expanded TILs from AML Patients Using Ex Vivo Cytotoxic Assays and In Vivo Homing Assays

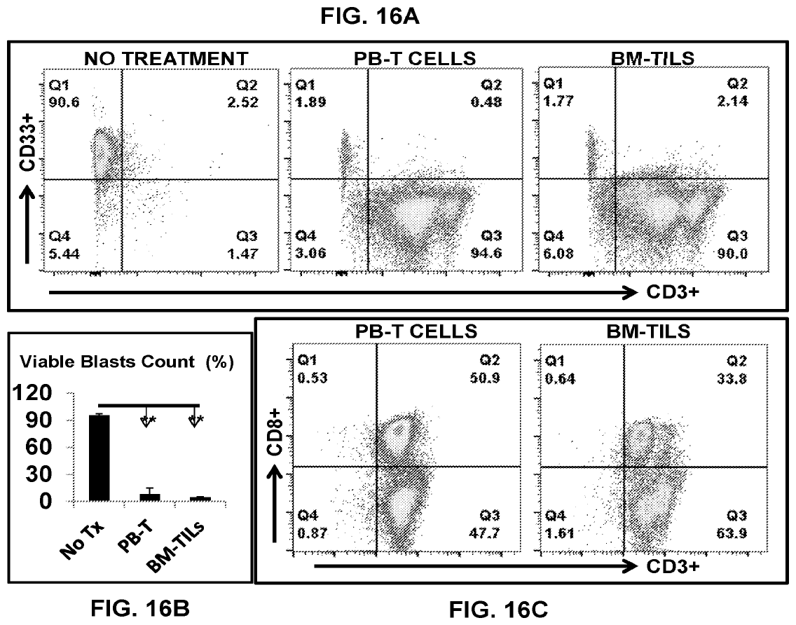

To examine the function of ex vivo expanded TILs, cytotoxic tests were performed. CD33, a surface biomarker, is expressed on leukemia blasts from the majority of AML patients. Thus, CD33⁺ AML blasts were isolated from BM samples by using CD33 antibodies with microbeads. Then, 2×10⁴-10⁵ autologous CD33⁺ blasts were co-cultured with 2×10⁵-10⁶ isolated and ex vivo expanded TILs (E:T ratio 10:1). After 18 hours, cells were collected and stained for FACS analysis. A significant decrease of viable CD33⁺ blast population were observed in TIL treatment versus the control of no treatment group (90.6% vs. 1.89%; p<0.01) (FIGS. 16A, 16B). TILs expanded from either PB or BM from the same patients were similarly effective against autologous blasts. FIGS. 16A-16C depict ex vivo cytotoxic tests of peripheral blood T cells (PB-T cells) and TILs from bone marrow (BM-TILs) from a patient having “quick growth” TILs (#14). FIG. 16A shows representative FACS plots showing the percentage of viable AML blasts in the cytotoxic test; The T cells versus blasts ratio equals 10:1. FIG. 16B is a graphical representation of the cumulative FACS percentage data of viable AML blasts. FIG. 16C shows representative FACS plots showing the percentage of viable CD8 percentage. Where applicable, data are means±SEM and were analyzed by Student t-test. **P<0.01, N=3.

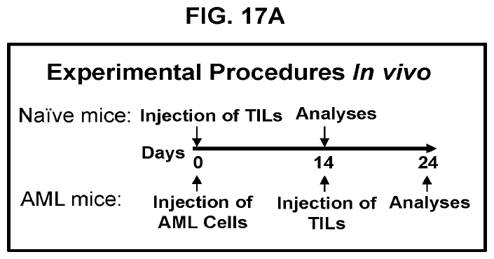

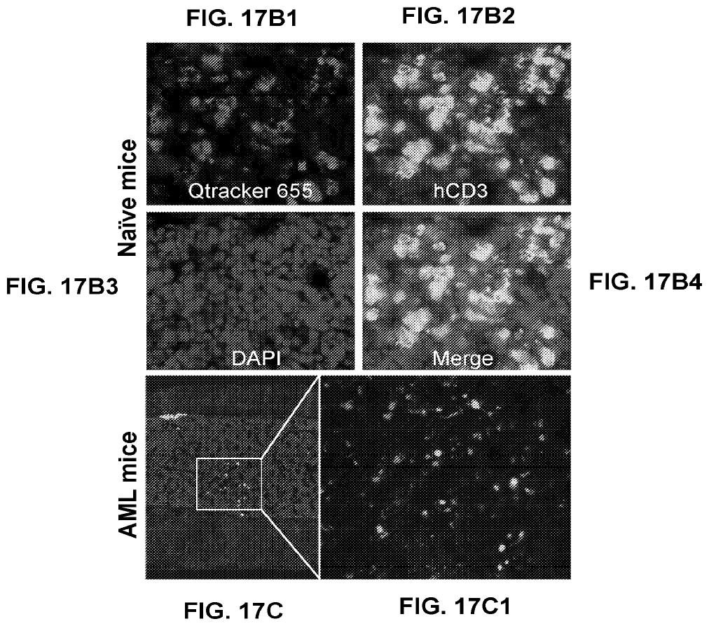

To investigate whether ex vivo expanded TILs will home to the BM and maintain their proliferation and functional capabilities in vivo, the following experiments were conducted. FIGS. 17A-17C depict transplantation of AML TILs in vivo. FIG. 17A is an illustration of the experimental procedures of transplanting TILs in vivo. TILs were pre-labeled with Qtracker 655 and then intravenously injected to naïve immune-deficient mice (NRG) (n=5). On day 14, mice were sacrificed and examined for the location of transplanted TILs. Transplanted TILs were found in the BMs of NRG mice, and continued to express CD3⁺ (FIG. 17B). FIG. 17B shows representative immunohistochemical images showing the colocalization of Qtracker 655⁺ TILs (red) with CD3 expression (green) in the bone marrow of naïve mice. DAPI: blue nuclei. AML blasts (GFP-labeled) were engrafted in NRG (n=5) mice followed by infusion of TILs (Qtracker 655 labeled). On day 24, mice were sacrificed for histology. TILs were found in BMs next to with GFP-labeled AML blasts (FIG. 17C). FIG. 17C shows representative Immunohistochemical images showing Qtracker 655⁺ TILs (red) located close to AML blasts (GFP-labeled, green) in the bone marrow of AML mice on day 10. The results demonstrate the presence of TILs in AML patients' bone marrow samples. Their phenotypic and functional features were characterized using immunophenotyping and cytotoxic assay.

To examine the ex vivo expandability of TILs for the possibility of autologous transplantation, a novel ex vivo culture system was developed to expand TILs from AML patient BM samples with low numbers of CD3⁺ T cells to clinical scales. Furthermore, it was immunophenotypically determined that these TILs expressed either CCR7⁺ CD95−/or CD62L⁺CD45RA⁺, which are makers for naive T cells. Some patients have high numbers of CD3⁺ TILs while others have low numbers of CD3⁺ TILs in their BM. The presence of naïve T cells is the hallmark of expandability of T cells, even in patients with low initial CD3⁺ TILs. Finally, the data demonstrate that TILs can cause cytotoxicity to autologous blasts ex vivo, can be engineered to express desirable genes, and are able to migrate to BM after being transplanted into immunodeficiency mice in vivo. An in vivo experiment showed that transplantation of expanded TILs is feasible and that IV injected cells can be tracked to the bone marrow. These ex vivo expanded TILs are likely to maintain their BM homing capability, proliferation and therapeutic capabilities in vivo. The in vivo data also suggested that primary TILs could be engineered to overexpress a desirable gene for therapeutic purpose (FIG. 12E). Thus, TILs could be used a vehicle for gene therapy for autologous transplantation to treat AML. The results suggest that BM derived TILs based cell therapies is a promising, novel therapeutic strategy for AML patients and should be further explored. Homing, proliferating, cytotoxic and therapeutic capabilities are compared between BM derived TILs with circulating TILs from peripheral blood (PB) ex vivo and in vivo transplantation studies. Reprogrammable TIL based immunotherapy is a new personalized immunotherapy for the treatment of AML and relapsed/refractory AML.

Materials and Methods

Human Samples. AML BM samples (Patients #1-10, Table 1) were obtained from Loma Linda University Cancer Center Biospecimen Laboratory (LLUCCBL). AML Peripheral Blood and BM samples (Patients #11-20, Table 1) were obtained from the City of Hope National Medical Center (COHNMC). All donor patients signed an informed consent form. Sample acquisition was approved by the Institutional Review Boards at the LLUMC and the COHNMC in accordance with an assurance filed with and approved by the Department of Health and Human Services, and it met all requirements of the Declaration of Helsinki.

TABLE 1
List of AML patients for the FACS screening and ex vivo/in vivo studies

| | | Disease | | | Cytogenetics | |
|---|---|---|---|---|---|---|
| No. | Diagnosis | Status | Age | Sex | (Karyotype) | Gene Mutation |
| 1 | AML | Newly | 32 | F | Normal | FLT3, CEBPA and NPM1: |
| | | Diagnosed | | | | NEGATIVE |
| 2 | AML | Newly | 70 | M | Normal | The molecular analyses of CEBPA, |
| | | Diagnosed | | | | FLT3 |
| | | | | | | and NPM1 mutations show only positive |
| | | | | | | for FLT3 internal tandem duplication |
| | | | | | | mutation (FLT3-ITD) |
| 3 | AML | Diagnosed | 35 | M | Normal | FLT3, CEBPA and NPM1: |
| | | | | | | NEGATIVE |
| 4 | AML | Newly | 59 | M | Normal | Molecular Markers |
| | | Diagnosed | | | | From OSH Quest Diagnostic |
| | | | | | | CEBPA negative |
| | | | | | | NPM mutation not detected |
| | | | | | | FLT3 ITD not detected |
| | | | | | | FLT3 TKD not detected |
| 5 | AML | Newly | 53 | M | inv(16)(p13.1q22) | FLT3, CEBPA and NPM1: |
| | | Diagnosed | | | MYH11/CBFB | NEGATIVE |
| 6 | AML | Diagnosed | 65 | M | Normal | FLT3 ITD (⁺), CEBPA (−), NPM 1 (⁺), c |
| | | | | | | kit (−), PML RARA |
| 7 | AML | Newly | 45 | M | Normal | CEBPA, DNMT3A, FLT3, IDH1/2, |
| | | Diagnosed | | | | KIT, KRAS, NRAS, RUNX1 TP53 |
| 8 | AML | Diagnosed | 38 | F | t(8;21) RUNX1- | Mutations noted in KRAS, NF1, and |
| | | | | | RUX1T1 | TP53; Negative for IDH 1, IDH2 and |
| | | | | | | FLT3, RUNX1 |
| 9 | AML | Diagnosed | 30 | M | Normal | Intermediate Risk (Wild type NPM1 |
| | | | | | | without FLT3-ITD without adverse risk |
| | | | | | | genetic lesions) |
| 10 | AML | Newly | 33 | F | Normal | DNMT3A, NRAS, NPM1 |
| | | Diagnosed | | | | |
| 11 | AML | Newly | 78 | M | Normal | TP53,U2AF1,ASXL1,RUNX1, FLT3- |
| | | Diagnosed | | | | ITD |
| 12 | AML | Newly | 53 | M | 46,XY,r(3)(p26q29),del | TP53 |
| | | Diagnosed | | | (5)(q22q35),der(7)t(7;?;3) | |
| | | | | | (q22;?;p11)[17] | |
| 13 | AML | Newly | 37 | F | Normal | WT1, FLT3-ITD |
| | | Diagnosed | | | | |
| 14 | AML | Newly | 40 | M | Normal | DNMT3A, IDH2, KRAS, NRAS,NPM1 |
| | | Diagnosed | | | | |
| 15 | AML | Newly | 72 | F | Normal | DNMT3A, TET2 |
| | | Diagnosed | | | | |
| 16 | AML | Newly | 67 | M | Normal | IDH2, NPM1 |
| | | Diagnosed | | | | |
| 17 | AML | Newly | 20 | M | 46,XY,inv(16) | KIT |
| | | Diagnosed | | | (p13.1q22.1)[23] | |
| 18 | AML | Newly | 63 | M | 46,XY,i(21)(q10)[20] | RUNX1, WT1 |
| | | Diagnosed | | | | |
| 19 | AML | Newly | 63 | F | Normal | NPM1, SF3B1 |
| | | Diagnosed | | | | |
| 20 | AML | Newly | 38 | F | Normal | FLT3, WT1, NPM1 |
| | | Diagnosed | | | | |

Mice. NRG (OD-Rag1^{null} IL2rg^{null}) mice were purchased from the Jackson Laboratory (Bar Harbor, ME) and housed in a specific pathogen-free animal facility at Loma Linda University (LLU). All mice were used at the age of 8 weeks. All experiments were performed in compliance with an Institutional Animal Care and Use Protocol approved by LLU Animal Care and Use Committee.

Isolation of TILs from AML patient bone marrow samples. CD3⁺ T cells from bone marrow mononuclear cells (BMMNC) were separated by using CD3 microbeads (Miltenyi Biotech, Germany) and a MiniMACS™ Separator with an MS Column according to the manufacturer's protocol. Selected CD3⁺ T cells were considered AML TILs.

Ex-Vivo Expansion of high number TILs by a Traditional T cell Protocol. CD3⁺ TILs were isolated from AML BMMNC by the pull-down through CD3 microbeads and magnetic separation. The non-CD3⁺ cells (feeder cells) were pre-treated with 10 mitomycin-C for 2 hours to arrest cell proliferation. CD3⁺ TILs and feeder cells were co-cultured at 37° C. and 5% CO₂ in a RPMI 1640 culture medium containing 10% fetal bovine serum (FBS, HyClone), 100 μg/ml penicillin/streptomycin, and Interleukin (IL-2) (1000 U/ml, Peprotech). Seeding cell density was 300 μl of 100,000 cells/ml in each well of 48-well-plates. For the maintenance of quickly expanded TILs, medium was changed every 2-3 days, and split cells at the ratio of 1:4 when reaching 80% confluent. TILs were stimulated with 30 ng/mL human anti-CD3 (OKT3, Biolegend). Around 10-14 days, cultures of TILs were started in 12-well-plates or T25 flasks for expansion of large amounts before further analyses.

Ex-Vivo Expansion of low number TILs by a Modified T cell Protocol. (1) Media with Cytokines: CD3⁺ T cells were cultured at 37° C. and 5% CO₂ in a RPMI 1640 culture medium containing 10% fetal bovine serum (FBS, HyClone) with penicillin/streptomycin (100 μg/ml), IL-2 (1000 U/ml, Peprotech), and Dynabeads® Human T-Activator CD3/CD28 (Gibco™) without feeder cells. (2) Timeline of TIL expansion: Stage 1 (Naïve TILs): The seeding cell density of CD3⁺ TILs was around 300 μl of 20,000 cells/ml in appropriate wells of 48-well-plates. Due to the low cell density of TILs, fresh media was added at a 1:1 ratio to each well every 2 days and mixed the cells/media. Based primarily on the growth of the TILs, media was changed every 5-7 days and split cells at the ratio 1:3. Stage 2 (Ready to grow): After 7 days, IL-7 (25 ng/ml, Peprotech) and IL-15 (25 ng/ml, Peprotech) were added to the media along with IL-2 (1000 U/ml). Every patient BMMNC sample was different; however, TILs were preferably raised in 48-well-plates for expansion to sufficient amounts during the beginning 10-14 days instead of in large wells and flasks. Stage 3 (Quickly expand and differentiate into T effectors): After 10-14 days, TILs grew very fast. Media change was performed every 2 days and split quickly expanded TILs at the ratio 1:4 to 1:8. Then, TILs were expanded in multiple 48 or 24 well-plates. Dynabeads® Human T-Activator CD3/CD28 was used once for re-stimulation of TILs.

Flow Cytometry (FACS). Expanded TILs were harvested and examined for the expression of cell surface biomarkers (CD) and intracellular proteins for T cells by multichromatic FACS. Briefly, about 1×10⁴˜10⁶ cells in 100 μl FACS buffer (PBS containing 1% FBS and 0.05% sodium azide) were stained with various fluorescence-conjugated antibodies specific for the desired cell surface proteins at 4° C. for 30 min. The surface-stained cells were then fixed and permeabilized using the appropriate reagents (e.g. the BD Pharmingen Cytofix/Cytoperm buffer) and stained with different fluorescence-conjugated antibodies specific for the desired intracellular proteins at 4° C. for 30 minutes in the permeabilizing buffer (e.g. the BD Perm/Wash buffer). Finally, the cells were washed twice in the permeabilizing buffer and twice in the FACS buffer before being analyzed on the BD FACSAria II. Data was analyzed using the FlowJo software (Treestar).

Cytotoxicity Assay. The cytotoxicity assays were performed by co-culturing engineered TILs with primary AML blasts from the same patient (isolated by CD33-microbeads pull-down) in 24-well plates. AML blasts from BMMNC were separated using APC anti-human CD33 antibody (Biolegend), anti-APC microbeads (Miltenyi Biotech, Germany), and a MiniMACS™ Separator with an MS Column. The ratio of autologous TILs to AML blasts were in the range of 5:1 to 10:1 according to a previous report [20]. After overnight incubation, cells were collected, stained, and processed for FACS assay of biomarkers including viability dyes (Invitrogen™) and CD33 according to manufacturers' protocols. Analyses and graphs will be generated using the GraphPad Prism software to evaluate significance.

Adoptive Cell Transplantation of engineered human AML cells and TILs in immune-deficient NRG mice. Ex vivo expanded TILs (2×10⁶ cells/mouse) were pre-labeled to be red fluorescent with Qtracker™ 655 (Molecular Probes) and intravenously (IV) injected into NRG mice through the tail vein. To help the engraftment, 10 mg/kg of Azacitidine was intraperitoneally injected one day before the injection. TILs-engrafted mice were sacrificed at different time points. In another experiment, AML cells (non-CD3⁺ cells from BMMNC) were transduced with GFP lentivirus to generate GFP⁺ AML cells. Fourteen days after transplantation of GFP⁺ AML cells (1×10⁶ cells/mouse), Qtracker™ 655 labelled TILs were IV injected into these AML NRG mice. The detailed protocol and plasmids for generating lentivirus and generating GFP⁺ AML cells can be found in our previous report [21]. On day 10 after TILs' engraftment, mice were sacrificed for FACS analyses. Immunofluorescent histology was performed to visualize TILs and GFP⁺ AML cells inside of the bone marrow.

Histology. Preparation of undecalcified frozen sections from bone tissues was performed according to the protocol reported in Xu, Y., et al., 2020 Transl Oncol 13(12): p. 100869. Briefly, specimens were fixed in 4% paraformaldehyde, freeze-embedded with an embedding medium (SCEM), and frozen in pentane cooled with liquid nitrogen. The frozen specimen block was fixed to the cryostat and trimmed with a disposable blade. The block's surface was then covered with a pressure sensitive adhesive film (Cryofilm) and cut into 10 μm-thick frozen sections which were stored at −20° C. The frozen sections were immunohistochemically stained and photographed for further analyses.

Reagents

Table 2 shows exemplary reagents that can be used according to the present disclosure.

TABLE 2
List of Reagents

| | | | | Species |
|---|---|---|---|---|
| Antibody/Reagents | Color | Cat. # | Company | Reactivity |
| CD3 | PE/Cyanine7 | 300420 | Biolegend | Human |
| CD3 | FITC | 300406 | Biolegend | Human |
| CD4 | APC | 17-0048-41 | eBioscience | Human |
| CD4 | PERCP | 300527 | Biolegend | Human |
| CD8 | PerCP/Cy5.5 | 302922 | Biolegend | Human |
| CD8 | FITC | 130-110-815 | Milteny Biotec | Human |
| CD95 | APC | 305611 | Biolegend | Human |
| CCR7 (CD197) | PE | 353203 | Biolegend | Human |
| CCR7 (CD197) | APC | 352313 | Biolegend | Human |
| CD62L | FITC | 304803 | Biolegend | Human |
| CD45RA | PE/Cyanine7 | 304125 | Biolegend | Human |
| PD-1 (CD279) | FITC | 367411 | Biolegend | Human |
| PD-1 (CD279) | PE | 12-2799-42 | eBioscience | Human |
| PD-L1 (CD274) | PerCP- | 46-5983-42 | eBioscience | Human |
| | eFluor710 | | | |
| CD33 | APC | 303408 | Biolegend | Human |
| Anti-APC Microbeads | | 130-090-855 | Milteny Biotec | |
| Annexin V | FITC | 640906 | Biolegend | |
| Viability Dye eFluor ™ 780 | | 65-0865-14 | eBioscience | |
| DAPI | | D9542-1MG | Sigma Aldrich | |
| Qtracker ™ 655 | | Q25029 | Molecular Probes | |
| CD3 MicroBeads | | 130-050-101 | Milteny Biotec | Human |
| Dynabeads ® Human T- | | 11161D | Gibco | Human |
| Activator CD3/CD28 | | | | |
| IL2 | | 200-02 | Peprotech | Human |
| IL7 | | 200-07 | Peprotech | Human |
| IL15 | | 200-15 | Peprotech | Human |
| KAWAMOTO Film Kit | | | Section-Lab Co.ltd, | |
| | | | Japan | |
| Azacitidine (AZA) | | | Celgene | |
| Nivolumab (Opdivo) | | | Bristol-Myers | |
| | | | Squibb | |

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

What is claimed is:

1. A method of treating acute myeloid leukemia in a subject, the method comprising the step of:
administering a tumor infiltrating lymphocytes-based adoptive cell therapy along with therapeutically effective amounts of an inhibitor of programmed death-1 (PD-1 inhibitor) and a hypomethylating agent, thereby treating acute myeloid leukemia in the subject.

2. The method of claim 1, wherein the hypomethylating agent is 5-azacytidine (AZA).

3. The method of claim 1, wherein the tumor infiltrating lymphocytes-based adoptive cell therapy comprises an administration of ex vivo expanded autologous PD-1-inhibited-tumor infiltrating lymphocytes.

4. The method of claim 1, wherein the tumor infiltrating lymphocytes-based adoptive cell therapy comprises an administration of ex vivo expanded bioengineered tumor infiltrating lymphocytes exposed to a PD-1 inhibitor.

5. The method of claim 4, wherein the ex vivo expanded tumor infiltrating lymphocytes are bioengineered to express 25-hydroxyvitamin D-1 alpha hydroxylase (CYP27B1).

6. The method of claim 1, wherein the PD-1 inhibitor is an anti-PD-1 antibody.

7. A method of treating acute myeloid leukemia in a subject, the method comprising the steps of:
isolating tumor infiltrating lymphocytes (TILs) from the subject;
culturing the isolated TILs in a culture system to produce cultured TILs; and
administering the cultured TILs to the subject, thereby treating acute myeloid leukemia in the subject.

8. The method of claim 7, wherein the TILs are isolated from a bone marrow sample from the subject.

9. The method of claim 7, wherein the TILs are isolated from a peripheral blood sample from the subject.

10. The method of any one of claims 7-9, wherein the TILs comprise CD3⁺ T cells.

11. The method of any one of claims 7-10, wherein the TILs comprise CCR7⁺CD95⁻ and/or CD62L⁺CD45RA⁺ T cells.

12. The method of claim 10, wherein the step of culturing the isolated TILs comprises increasing the number of CD3⁺ TILs by about a 1000-fold relative to a sample from the subject.

13. The method of any one of claims 7-12, wherein the step of culturing the isolated TILs comprises exposing the isolated TILs to a PD-1 inhibitor.

14. The method of claim 13, wherein the PD-1 inhibitor is an anti-PD-1 antibody.

15. The method of any one of claims 7-14, wherein the step of culturing the isolated TILs comprises increasing the ratio of CD8⁺ TILs.

16. The method of any one of claims 7-15, further comprising the step of:
bioengineering the isolated TILs to increase expression or function of a CYP27B1 gene.

17. The method of any one of claims 7-16, further comprising the step of:
bioengineering the isolated TILs to reduce expression or function of a PD-1 gene.

18. The method of any one of claims 7-17, further comprising the step of:
bioengineering the isolated TILs to increase expression or function of one or more of CD3, CCR7, CD62L, CD45RA, CD95, CD127, CD27, and CD28.

19. The method of any one of claims 7-18, further comprising administering a therapeutically effective amount of a PD-1 inhibitor and a hypomethylating agent to the subject, thereby treating acute myeloid leukemia in the subject.

20. The method of claim 19, wherein the hypomethylating agent is 5-Azacytidine (AZA).
